(19) United States
(12) Patent Application Publication
(10) Pub. No.: US 2003/0003454 A1
(43) Pub. Date: Jan. 2, 2003

(54) Methods and kits for determining a risk to develop cancer, for evaluating an effectiveness and dosage of cancer therapy and for correlating between an activity of a DNA repair enzyme and a cancer

(75) Inventors: Zvi Livneh, Rehovot (IL); Tamar Paz-Elizur, Rehovot (IL); Sara Blumenstein, Rehovot (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(73) Assignee: Yeda Research and Development Co. Ltd. at The Weizmann Institute of Science

(21) Appl. No.: 09/815,015

(22) Filed: Mar. 23, 2001

(51) Int. Cl. C12Q 1/68; G01N 33/574

(52) U.S. Cl. 435/6; 435/7.23

(57) ABSTRACT

Methods and kits for (i) determining a risk of a subject to develop cancer; (ii) evaluating an effectiveness and dosage of cancer therapy administered to a cancer patient; and (iii) determining a presence of correlation or non-correlation between an activity of at least one DNA repair enzyme and at least one cancer, are disclosed.

### FIELD AND BACKGROUND OF THE INVENTION

The present invention relates to the field of diagnosis and prognosis. More particularly, the present invention relates to methods and kits for (i) determining a risk of a subject to develop cancer; (ii) evaluating an effectiveness and preferred dosage of cancer therapy administered to a cancer patient; and (iii) determining a presence of correlation or non-correlation between an activity of at least one DNA repair enzyme and at least one cancer.

The DNA in each cell of a body is constantly subjected to damage caused by both internal (e.g., reactive oxygen species) and external DNA damaging agents (e.g., sunlight, X-and γ-rays, smoke) (Friedberg, et al., 1995). Most lesions are eliminated from DNA by one of several pathways of DNA repair (Friedberg, et al., 1995, Hanawalt, 1994, Modrich, 1994, Sancar, 1994). When unrepaired DNA lesions are replicated, they cause mutations because of their miscoding nature (Echols and Goodman, 1991, Livneh, et al., 1993, Strauss, 1985). The occurrence of such mutations in critical genes, e.g., oncogenes and tumor suppressor genes, may lead to the development of cancer (Bishop, 1995, Vogelstein and Kinzler, 1993, Weinberg, 1989). Indeed, DNA repair has emerged in recent years as a critical factor in cancer pathogenesis, as a growing number of cancer predisposition syndromes have been shown to be caused by mutations in genes involved in DNA repair and the regulation of genome stability. These include Xeroderma Pigmentosum (Weeda, et al., 1993), Hereditary nonpolyposis colon cancer (Fishel, et al., 1993, Leach, et al., 1993, Modrich, 1994, Parsons, et al., 1993), Ataxia Telangiectasia (Savitsky, et al., 1995), Li-Fraumeni syndrome (Srivastava, et al., 1990), and the BRCA1 (Gowen, et al., 1998, Scully, et al., 1997) and BRCA2 genes (Connor, et al., 1997, Patel, et al., 1998, Sharan, et al., 1997). In these cases, which represent a minority of the cancer cases, gene mutations have caused malfunction, leading to a strong reduction in DNA repair.

A possible extension of the role of DNA repair in hereditary cancer, would be a role for DNA repair in sporadic cancer. Several studies suggested that inter-individual variability in DNA repair correlates with variation in cancer susceptibility, with low repair correlated to higher cancer risk (Athas, et al., 1991, Helzlsouer, et al., 1996, Jyothish, et al., 1998, Parshad, et al., 1996, Patel, et al., 1997, Sagher, et al., 1988, Wei, et al., 1996, Wei, et al., 1993, Wei, et al., 1994).

8-OxoG is formed in DNA by two major pathways: (a) Modification of guanine in DNA by reactive oxygen species formed by intracellular metabolism, oxidative stress, cigarette smoke, or by radiation (Asami, et al., 1997, Gajewski, et al., 1990, Hutchinson, 1985, Leanderson and Tagesson, 1992). (b) Incorporation into DNA by DNA polymerases of 8-oxo-dGTP, which is formed by oxidation of intracellular dGTP (Maki and Sekiguchi, 1992). Once in DNA, 8-oxoG is replicated by DNA polymerases with the misinsertion of dAMP, causing characteristic GC→TA transversions (Shibutani, et al., 1991, Wood, et al., 1990). When the modified dGTP is used as a substrate by DNA polymerases, it is often misinserted opposite an A in the template, causing AT→CG transversions (Pavlov, et al., 1994).

The major route for removing 8-oxoG from DNA is base excision repair, initiated by 8-oxoguanine DNA N-glycosylase, product of the OGGl gene (in humans termed also hOGG; (Aburatani, et al., 1997, Arai, et al., 1997, Bjoras, et al., 1997, Radicella, et al., 1997, Roldan-Arjona, et al., 1997, Rosenquist, et al., 1997). The OGGl gene was recently knocked-out in mice, such that the effects on carcinogenesis can now be examined in this organism (Klungland, et al., 1999, Minowa, et al., 2000). Expression of the E. coli enzyme in Chinese hamster cells reduced 4-fold the mutagenicity of γ radiation (Laval, 1994), indicating that the repair of 8-oxoG is important in negating the mutagenic activity of γ radiation. The following observations associate OGGl with cancer: (i) OGGl was mapped to chromosome 3p25, a site frequently lost in human lung and kidney cancers (Arai, et al., 1997, Audebert, et al, 2000, Ishida, et al., 1999, Lu, et al., 1997, Wikman, et al., 2000). (ii) OGGl was found to be mutated in 2 out of 25 lung tumors (Chevillard, et al., 1998), and in 4 out of 99 renal tumors (Audebert, et al., 2000). (iii) OGGl was found to be mutated in a leukemic cell line (Hyun, et al., 2000), in stomach cell line, and in a gastric cell line (Shinmura, et al., 1998). (iv) Analysis of p53 mutations in human lung, breast, and kidney tumors revealed a substantial occurrence of GC→TA mutations, a mutation type produced by unrepaired 8-oxoG (Hernandez-Boussard, et al., 1999).

Since preventive measures which reduce the risk of developing cancer, such as, but not limited to, the use of anti-oxidants, diet, avoiding cigarette smoking, refraining from occupational exposure to cancer causing agents, are known and further since periodic testing and therefore early detection of cancer offers improved cure rates, there is a great need for, and it would be highly advantageous to have methods and kits for determining a risk of a subject to develop cancer.

Since the effectiveness of cancer therapy depends on the sensitivity of cells to genotoxic (mutageic) agents, there is a great need for, and it would be highly advantageous to have methods and kits for evaluating an effectiveness and preferred dosage of cancer therapy administered to a cancer patient.

There is also a great need for, and it would be highly advantageous to have methods and kits for determining a presence of correlation or non-correlation between an activity of at least one DNA repair enzyme and at least one cancer, so as to allow to determine a risk of a subject to develop cancer and to evaluate an effectiveness and preferred dosage of cancer therapy administered to a cancer patient.

### SUMMARY OF THE INVENTION

According to one aspect of the present invention there is provided method of determining a risk of a subject to develop cancer, the method comprising determining a level of activity of a DNA repair/damage preventing enzyme in a tissue of the subject, and, according to the level, determining the risk of the subject to develop the cancer.

According to another aspect of the present invention there is provided a method of determining a risk of a subject to develop cancer, the method comprising determining (a) a presence or absence of exposure to environmental conditions, such as smoking and occupational exposure to smoke or ionizing radiation, associated with increased risk of developing cancer; and (b) a level of activity of a DNA repair/damage preventing enzyme in a tissue of the subject; and according to the presence or absence and the level, determining the risk of the subject to develop the cancer.

According to still another aspect of the present invention there is provided a method of determining a presence of correlation or non-correlation between an activity of at least one DNA repair/damage preventing enzyme and at least one cancer, the method comprising determining a level of activity of at least one DNA repair/damage preventing enzyme in tissue derived from a plurality of cancer patients and a plurality of apparently normal individuals, and, according to the level determining the correlation or non-correlation between the activity of the at least one DNA repair/damage preventing enzyme and the at least one cancer.

According to further features in preferred embodiments of the invention described below, the cancer is selected from the group consisting of lung cancer, blood cancers, colorectal cancer, breast cancer, prostate cancer, ovary cancer and head and neck cancer.

According to still further features in the described preferred embodiments the tissue is selected from the group consisting of blood cells, scraped cells and biopsies.

According to still further features in the described preferred embodiments the DNA repair/damage preventing enzyme is selected from the group consisting of a DNA N-glycosylase, deoxyribose phosphate lyase and AP endonuclease.

According to still further features in the described preferred embodiments the DNA N-glycosylase is selected from the group consisting of Uracil DNA glycosylase, hSMUG1, hMBD4, Mismatch-specific thymine/uracil glycosylase, Methylpurine DNA glycosylase, hNTH1, Adenine-specific mismatch DNA glycosylase and 8-oxoguanine DNA glycosylase.

According to still further features in the described preferred embodiments the risk is expressed as a fold risk increase as is compared to a normal, apparently healthy, population.

According to still further features in the described preferred embodiments the risk is expressed in enzyme specific activity units.

According to still further features in the described preferred embodiments the risk is expressed as a magnitude of a scale.

According to still further features in the described preferred embodiments determining the level of activity of the DNA repair/damage preventing enzyme is effected using a DNA substrate having at least one lesion therein.

According to still further features in the described preferred embodiments the at least one lesion is at a predetermined site in the DNA substrate.

According to still further features in the described preferred embodiments the lesion is selected from the group consisting of uracil, 5-fluorouracil, 5-hydroxyuracil, isodialuric acid, alloxan, uracil or thymine in U/TpG:5meCpG, uracil (U:G), 3,N⁴-ethenocytosine, (eC:G), T (T:G), 3-methyladenine, 7-methyladenine, 3-methylguanine, 7-methylguanine, hypoxanthine, 1, N6-ethenoadenine, 1,N2-ethenoguanine, thymine glycol, cytosine glycol, dihydrouracil, formamidopyrimidine urea, adenine from A:G; A:8-oxoG; C:A, 2-hydroxyadenine, 2,5-amino-5-formamidopyrimidine, 7,8-dihydro-8-oxoguanine and abasic site.

According to still further features in the described preferred embodiments the substrate includes at least two different lesions of at least two types.

According to still further features in the described preferred embodiments the substrate includes a single lesion.

According to still further features in the described preferred embodiments the substrate includes at least two different lesions of a single type.

According to still further features in the described preferred embodiments the subject is known to be, or is about to be, exposed to environmental conditions associated with increased risk of developing cancer.

According to yet another aspect of the present invention there is provided a method of predicting the efficacy of a mutagenic anti-cancer treatment, such as chemotherapy and/or radiotherapy, in a subject, the method comprising determining a level of activity of a DNA repair/damage preventing enzyme in a tissue of the subject, and, according to the level, predicting the efficacy of the mutagenic anti-cancer treatment in the subject.

According to still another aspect of the present invention there is provided a method of selecting dosage of a mutagenic anti-cancer treatment, such as chemotherapy and/or radiotherapy, for treating a subject, the method comprising determining a level of activity of a DNA repair/damage preventing enzyme in a tissue of the subject, and, according to the level, selecting dosage of the mutagenic anti-cancer treatment for treating the subject.

According to an additional aspect of the present invention there is provided a kit for determining a level of activity of a DNA repair/damage preventing enzyme in a tissue of a subject, the kit comprising, a package including, contained in sealable containers, a DNA substrate having at least one lesion therein and a reaction buffer.

According to further features in preferred embodiments of the invention described below, the kit, further comprising test tubes for separating lymphocytes.

According to still further features in the described preferred embodiments the test tubes are prepackaged with an anti-coagulant.

According to still further features in the described preferred embodiments the kit further comprising a liquid having a specific gravity selected effective in separating lymphocytes from red blood cells via centrifugation.

According to still further features in the described preferred embodiments the kit further comprising a solution having osmolarity selected effective in lysing red blood cells.

According to still further features in the described preferred embodiments the kit further comprising a protein extraction buffer.

According to still further features in the described preferred embodiments the kit further comprising reagents for conducting protein determinations.

According to still further features in the described preferred embodiments the kit further comprising a purified DNA repair/damage preventing enzyme, which serves as a control for such activity.

The present invention successfully addresses the shortcomings of the presently known configurations by providing, methods, kits and reagents useful in determining a risk of a subject to develop cancer and for evaluating an effectiveness and individual dosage of cancer therapy administered to a cancer patient.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1a shows an outline of an OGGA nicking assay according to the present invention. In the assay a 32 base pair synthetic DNA is cleaved at an 8-oxoG lesion (indicated by a circle), generating, after denaturation, a radiolabeled 17-mer. The asterisk represents a radiolabeled phosphate group.

FIGS. 1b-c represent a time course of the OGGA nicking assay of the present invention, performed under standard conditions, with a protein extract prepared from peripheral blood lymphocytes from a healthy donor. FIG. 1b shows a phosphorimage of the reaction products fractionated by urea-PAGE, and FIG. 1c shows the quantification of the images. GO, the DNA substrate with a site-specific 8-oxoG; C, a control substrate with a G instead of 8-oxoG.

FIGS. 2a-b show protein titration in the OGGA nicking assay. The assay was performed under standard conditions, with the indicated amounts of protein extract prepared from peripheral blood lymphocytes from a healthy donor. FIG. 2a shows a phosphorimage of the reaction products fractionated by urea-PAGE, and FIG. 2b shows the quantification of the images. GO, the DNA substrate with a site-specific 8-oxoG; C, a control substrate with a G instead of 8-oxoG.

FIGS. 3a-b show analysis of the specificity of the OGGA nicking assay of the present invention. The assay was performed under standard conditions, except that the reaction mixture contained 2 pmol of radiolabeled substrate containing 8-oxoG, and the indicated amounts of unlabeled competing DNA. FIG. 3a shows a phosphorimage of the reaction products fractionated by urea-PAGE, and FIG. 3b shows the quantification of the images. The protein extract was from a healthy donor Hx, G and GO represent unlabeled competing DNAs, which were similar to the radiolabeled substrate, and contained either hypoxanthine, guanine or 8-oxoG in the same location.

FIG. 4 shows the OGGA distribution in healthy individuals. The OGGA nicking assay of the present invention was performed with blood samples from 82 healthy donors. OGGA≦5.5 is defined as Low (less than 5 % of the healthy population) OGGA>5.5 is defined as Normal.

FIG. 5 shows a comparison of OGGA in males and females. The OGGA distribution of the 82 individuals shown in FIG. 4, was plotted separately for males (N=35) and females (N=47).

FIG. 6 shows a comparison of OGGA in smokers and non-smokers. The OGGA distribution of the 82 individuals shown in FIG. 4, was plotted separately for smokers (N=34) and non-smokers (N=45). Three individuals which were former smokers were excluded from the graph.

FIG. 7 shows a comparison of OGGA in two age groups. The OGGA distribution of the 82 individuals shown in FIG. 4, was plotted separately for ages 20-49 (N=35) and 50-80 (N=47)

FIGS. 8a-c show OGGA in apparently healthy individuals and in patients with breast cancer or chronic lymphocytic leukemia (CLL). FIG. 8a—OGGA distribution of the control group of 82 healthy individuals (see FIG. 4), and of 31 breast cancer patients (FIG. 8b), and 19 CLL patients (FIG. 8c).

FIGS. 9a-b show OGGA in apparently healthy individuals and in patients with lung cancer (NSCLC). FIG. 9a—OGGA distribution of the control group of 82 healthy individuals (see FIG. 4), and of 54 lung cancer (NSCLC) patients (FIG. 9b).

FIGS. 10a-b show OGGA in apparently healthy individuals and in lymphoma patients. FIG. 10a—OGGA distribution of the control group of 82 healthy individuals (see FIG. 4), and of 18 lymphoma patients (FIG. 10b).

FIGS. 11a-b show OGGA in apparently healthy individuals and in patients with colorectal cancer FIG. 11a—OGGA distribution of the control group of 82 healthy individuals (see FIG. 4), and of 16 colorectal cancer patients (FIG. 11b).

FIGS. 12a-b are schematic representations of monomolecular (FIG. 12a) and plurimolecular (FIG. 12b) universal substrates in accordance with the teachings of the present invention.

### DESCRIPTION OF THE PREFERRED EMBODIMENTS

The present invention is of methods and kits which can be used for (i) determining a risk of a subject to develop cancer; (ii) evaluating an effectiveness and dosage of cancer therapy administered to a cancer patient; and (iii) determining a presence of correlation or non-correlation between an activity of at least one DNA repair/damage preventing enzyme and at least one cancer.

The principles and operation of a method and kit according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

While conceiving the present invention it was hypothesized that inter-individual variations in DNA repair/damage preventing activity modulate susceptibility of developing cancer.

While reducing the present invention to practice an experimental system which is easily adaptable to clinical use was developed, such that a defined DNA repair activity can now be used in determining cancer risk, and be utilized as a tool in cancer prevention, early detection and prognosis. Since the repertoire of DNA lesions is very large, at present experimental focus was given to an abundant and mutagenic DNA lesion, 8-oxoguanine (also termed 7, 8-dihydro 8-oxoguanine or 8-hydroxyguanine; dubbed 8-oxoG). However, other mutagenic DNA lesions, such as, but not limited to, those listed in Table 1 below, can be similarly used to implement the methods of the invention, following suitable adaptation.

Thus, while reducing the present invention to practice, whether inter-individual variations in the activity of OGG, correlate with increased susceptibility to several types of cancers was studied. A lower repair activity might lead to an increased load of DNA lesions, and therefore to increased mutation rate, and earlier occurrence of cancer. Similarly, a lower repair activity renders cancer cells more susceptible to cancer therapy, which is genotoxic by nature. It should be noted that different types of DNA repair may be critical in different types of cancer. The present invention is exemplified, in a non-limiting fashion, with respect to the removal from DNA of a specific type of mutagenic lesion, 8-oxoG, by the activities of one or more DNA N-glycosylase repair enzymes, present in protein extracts from peripheral blood lymphocytes. Unlike DNA polymorphism and gene mutations, which are variations at the DNA level only, the inter-individuals variability in DNA repair enzyme activity is expected to be affected by multiple parameters. These include, for example, mutations in the coding sequence, the level of expression, the stability of the protein (which in turn depends on variations in processes controlling proteolysis), and the possible presence of inhibitors and stimulatory factors. These numerous variations culminate into the final activity of the enzyme(s), which may vary considerably among individuals. This approach has the advantage that it is simple, and allows highly accurate and reproducible measurement of DNA repair activity. It should be emphasized that the observed enzymatic activity might represent several human enzymes, that have a similar repair activity. With respect to 8-oxoG, so far only OGGl was found in humans, however, one cannot exclude the possibility that there are additional enzymes with remove 8-oxoG from DNA. If present in the whole cell protein extract, such enzymes will contribute to the final activity measured in an assay provided by the present invention.

Thus, the present invention is herein exemplified with respect to the use of the level of the DNA repair enzymatic activity of DNA N-glycosylase(s) directed toward 7, 8 dihydroxy 8-oxoguanine (8-oxoguanine DNA N-glycosylase activity; OGG), as a risk factor for lung cancer, lymphomas, and colorectal cancer. The enzymatic activity is measured in a protein extract extracted from peripheral blood lymphocytes and is referred to herein interchangeably as the OGGA nicking assay, OGGA assay or OGGA test.

Using the OGGA test, a study was conducted on 220 individuals: 82 healthy individuals, and a total of 138 cancer patients as follows: 54 lung cancer (NSCLC) patients, 31 breast cancer patients, 18 lymphoma patients, 19 CLL patients, and 16 colorectal cancer patients. The following results were found.

The mean OGGA in healthy individuals of ages 20-49 (7.62±0.87; N=35) was similar to healthy individuals of ages 50-80 (7.25±1.11; N=47). The difference was not statistically significant (P=0.107).

The mean OGGA in healthy men (7.71±0.95; N=35) was slightly higher than in healthy women (7.19±11.03; N=47), the difference being statistically significant (P=0.019).

The mean OGGA in smokers (7.38±1.00; N=34) was similar to that of non-smokers (7.39±1.06; N=45), indicating that the smoking status had a negligible effect on OGGA.

The mean OGGA in lung cancer patients (6.19±1.63; N=54) was significantly lower than in healthy individuals (7.41±1.02; N=82), with P=0.0001.

Low repair OGGA indexes is defined as≦5.5 units/μg protein, representing<5% of the healthy individuals. Normal repair is defined as OGGA>5.5 units/μg protein. After adjustment for age and smoking status, lung cancer patients were 14 times more likely than the healthy controls to have a low OGGA index (Odds Ratio 13.89; 95% confidence interval, 2.53-76.9). This indicates that a low OGGA index is a strong risk factor in lung cancer.

Smokers with low OGGA index have a 25.8-fold higher risk for developing lung cancer compared to smokers with normal OGGA. For non-smokers, the increased risk is marginal (2.39-fold). This means that low OGGA sensitizes individuals to smoking-induced lung cancer.

The mean OGGA in lymphoma patients (6.16±1.84; N=18) was significantly lower than in healthy individuals (7.41±1.02; N=82), with P=0.01 17.

After adjustment for age, lymphoma patients were 12 times more likely than the healthy controls to have a low OGGA index (Odds Ratio 12.66; 95% confidence interval, 2.66-58.82). This indicates that a low OGGA index is a strong risk factor in lymphoma.

The data shows that OGGA was low in 2 out of 16 (12%) colorectal cancer patients (compared to 3/82 i.e., 3.7% among healthy individuals), indicating that low OGGA is a risk factor in colorectal cancer.

OGGA distribution was normal in breast cancer patients and in CLL patients, indicating that OGGA is not a risk factor in these types of cancer.

It will be appreciated that the OGGA and similar tests for other DNA repair activities can be used for screening individuals for purposes of prevention, early diagnosis and prognosis of cancers. These uses will be described in more detail below.

The following provides examples:

(i) Screening for smokers who have low OGGA in order to prevent lung cancer.

Although 87% of lung cancer patients are smokers, only 2.5% of all smokers develop lung cancer. The results presented herein suggest that smokers who have Low OGGA have a 25.8-fold higher risk than smokers with Normal OGGA for developing lung cancer. The simplest explanation for this finding is that smokers with Low OGGA in peripheral blood lymphocytes have a lower OGGA also in their lungs, and that this causes their lungs to cope less well with the DNA damage resulting from cigarette smoking. This is a classical example in which the risk of developing cancer is a combination of genetic factors (level of DNA repair) and external factors (cigarette smoking). Such individuals may be persuaded to quit smoking. Their genetic predisposition might be harmless, unless exposed to a DNA damaging agent (e.g., cigarette smoke). Such a screen will be effective as a preventive means against lung cancer, and will lead eventually to a decrease in the incidence of lung cancer.

(ii) Avoiding occupational hazard.

A considerable amount of people work in places which deal with radiation or with smoke. These include radiology departments in hospitals, nuclear industry, nuclear reactors, army personal dealing with nuclear weapons, etc. These people can be tested for OGGA, as a mandatory test, for their own safety. Individuals with Low OGGA might have an increased probability of developing cancer in such places, since ionizing radiation and smoke each produce 8-oxoG. Such individuals will be advised to seek an alternative working environment.

(iii) Using the OGGA index as a prognostic marker for cancer therapy.

Cancer therapy relies heavily on chemicals and radiation. These agents act, in most cases, by inflicting massive DNA damage, which leads to selective killing of the rapidly dividing cancer cells. The problem with such therapeutic agents is that they damage, or kill, also non-cancer cells. Knowing the level of OGGA in a cancer patient, may be used as a marker to estimate the prognosis of a particular therapeutic treatment.

(iv) Screening for susceptibility to lymphoma or colorectal cancers.

OGGA can be used to screen individuals for susceptibility to lymphomas or colorectal cancer.

(v) Early detection of cancer.

Individuals with low OGGA (e.g., smokers with low OGGA who would not quit smoking) can be advised to undergo periodical follow-ups, in order to enable early detection of lung cancer.

Thus, according to one aspect of the present invention there is provided a method of determining a risk of a subject to develop cancer. The method according to this aspect of the present invention is effected by determining a level of activity of a DNA repair/damage preventing enzyme in a tissue of the subject, and, according to the level, determining the risk of the subject to develop the cancer.

According to another aspect of the present invention there is provided a method of determining a risk of a subject to develop cancer. The method according to this aspect of the present invention is effected by determining a presence or absence of exposure to environmental conditions, such as smoking and occupational exposure to smoke or ionizing radiation, associated with increased risk of developing cancer; and determining a level of activity of a DNA repair/damage preventing enzyme in a tissue of the subject; and according to the presence or absence and the level, determining the risk of the subject to develop the cancer.

The present invention is useful in determining a risk of a subject to develop cancer, whereby any type of cancer is subject to risk determination by way of implementing the method of the invention. It is well known that all cancers arise from DNA mutations and that the progress of a specific cancer from a primary tumor to a metastatic tumor, reflects clonal selection of cancer cells that accumulate mutations as they develop and turn more cancerous (e.g., proliferate more rapidly, escape proliferation control, acquire autosignalling behavior, induce angiogenesis, etc.) and more metastatic. This process is subject to variations depending on the specific genes involved in the development and progression of different cancers. It is therefore expected that different in vivo DNA repair/damage preventing activities are required to prevent the formation of different cancers. Also, the level of exposure of body tissues to genotoxic agents such as smoke and radiation, differs. Since different types of genotoxic agents cause different types of DNA lesions, it is again expected that different in vivo DNA repair/damage preventing activities are required to prevent the formation of different cancers.

The results obtained while reducing the present invention to practice are in agreement with the above, as low OGGA was found to be associated with some, but not all cancers tested. However, assays similar to the OGGA assay described herein can be readily developed for correlating other cancers with one or more DNA lesions, some of which are listed in Table 1 below.

In effect, all known cancers can be evaluated by finding correlation or non-correlation between the occurrence thereof and the occurrence of low DNA repair/damage preventing activity of certain types. When positive correlation is identified, a predictive risk determination assay can be readily implemented.

Thus, according to an aspect of the present invention there is provided a method of determining a presence of correlation or non-correlation between an activity of at least one DNA repair/damage preventing enzyme and at least one cancer. The method according to this aspect of the invention is effected by determining a level of activity of at least one DNA repair/damage preventing enzyme in tissue derived from a plurality of cancer patients and a plurality of apparently normal individuals, and, according to the level determining the correlation or non-correlation between the activity of the at least one DNA repair/damage preventing enzyme and the at least one cancer. This aspect of the invention is exemplified herein with respect to a single DNA repair enzyme activity (8-oxoguanine DNA glycosylase) using a suitable substrate having a single lesion therein, for a plurality of cancers, for some correlation was found, whereas for other, non-correlation was found.

Thus, the methods of determining a risk of a subject to develop cancer described herein can be implemented for a variety of cancers, including, but not limited to, lung cancers, e.g., small-cells lung cancer and non-small cells lung cancer, blood cancers, e.g., lymphomas and leukemias, including, for example, Hodgkin's lymphoma, non-Hodgkin's lymphoma, acute lymphocytic leukemia, acute myelocytic leukemia, chronic lymphocytic leukemia, chronic myelogenous leukemia and the like, colorectal cancer, breast cancer, prostate cancer, ovary cancer, malignant melanoma, stomach cancer, pancreas cancer, urinary cancer; uterus cancer, bone cancer, liver cancer, thyroid cancer, brain cancer; head and neck cancer, including, for example, salivary carcinoma, laryngeal carcinoma and head and neck cancer

DNA repair/damage preventing activity can be measured in extracts of different body tissues or cells, which may be collected from a testee by known methods. Blood cells, scraped cells (e.g., mouth or skin scrapes) and biopsies are good examples as such tissues are routinely removed from subjects for diagnostics.

Several types of DNA repair/damage preventing activities can be assayed according to the present invention, e.g., DNA N-glycosylase, nucleotide pool sanitizing activity (dNTPase activity, e.g., 8-oxodGTPase), AP endonuclease and deoxyribose phosphate lyase (of DNA polymerase β).

An assay for determining the activity of a DNA N-glycosylase is described and exemplified herein with respect to 8-oxoguanine DNA glycosylase. In this respect it is convenient to monitor the nicking activity of DNA N-glycosylase towards DNA substrates including one or more lesion.

An assay for monitoring the activity of 8-oxodGTPase is, for example, as described by Mo et al. [Mo, J.-Y., Maki, H. and Sekiguchi, M. (1992) Proc. Natl. Acad. Sci. USA 89, 11021-11025]. Thus, 8-oxodGTPase activity can be assayed by measuring the hydrolysis of α-³²P-labeled 8-oxodGTP to 80 μg/ml oxodGMP. The reaction mixture (12.5 μl) contains 20 mM Tris-HCl (pH 8.0), 4 mM MgCl₂, 40 mM NaCl, 20 μM α-³²P-labeled 8-oxodGTP, 80 μg/ml bovine serum albumin, 8 mM dithiothreitol, 10% glycerol, and a protein extract. The reaction is executed at 30° C. for 20 minutes. Thereafter, an aliquot (2 μl) from the reaction mixture is spotted onto a PEI-cellulose TLC plate, and the mixture is fractionated with a solution containing 1 M LiCl for 1 hour. The spots on the TLC plate are then visualized and quantified by phosphorimaging. The preparation of 8-oxodGTP is described in Mo et al., ibid.

An assay for monitoring the activity of AP endonuclease is, for example, as described by Wilson III, et al. [Wilson III, D. M., Takeshita, M., Grollman, A. P., Demple B. (1995) Incision activity of human apurinic endonuclease (Ape) at abasic site analogs in DNA. J. Biol. Chem. 270, 16002-16007]. The reaction mixture (10 μl) contains 50 mM Hepes-KOH pH 7.5, 50 mM KCl, 100 μg/ml bovine serum albumin, 10 mM MgCl₂, 0.05% Triton X-100, 2 pmol of a the DNA substrate and a protein extract. Reactions are performed at 37° C. for 5-30 minutes, after which the reaction products are fractionated by urea-PAGE, to separate the intact and incised DNA strands. The activity is deduced from the extent of cleavage of the substrate. The preparation of the substrate is described in the same reference (Wilson III et al, ibid.).

An assay for monitoring the activity of deoxyribose phosphate lyase (dRPase) is, for example, as described by Prasad et al. [Prasad, R., Beard, W. A., Strauss, P. R. and Wilson, S. H. (1998) Human DNA polymerase β deoxyribose phosphate lyase. Substrate specificity and catalytic mechanism. J. Biol. Chem. 273, 15263-15270]. Deoxyribose phosphate lyase (dRPase) activity can be assayed by following the removal of deoxyribose phosphate from a ³²P 3′ end-labeled duplex oligonucleotide containing a site-specific 5′-incised abasic site. The reaction mixture (10 μl) contains 50 mM Hepes pH 7.4, 2 mM dithiothreitol, 5 mM MgCl₂, 20 nM ³²P-labeled duplex oligonucleotide with a site specific abasic site (pre-incised at the 5′ with AP endonuclease), and a protein extract. The reaction is carried out at 37° C. for 15 minutes. After the reaction is terminated, the product is stabilized by the addition of NaBH₄ to a final concentration of 340 mM, and incubated for 30 minutes at 0° C. The DNA is then ethanol precipitated and fractionated by urea-PAGE. The activity of the dRPase is deduced from the extent of formation of the shorter reaction product. The preparation of the DNA substrate is described in the same reference (Prasad et al, ibid.).

Table 1 below lists examples of DNA repair enzymes, the genes encoding same and the DNA lesion(s) they recognize:

TABLE 1

| Enzyme | | Gene | Substrate |
|---|---|---|---|
| 1. | Uracil DNA glycosylase | UNG1, 2 | uracil, 5-fluorouracil, 5-hydroxyuracil |
| | | | isodialuric acid, alloxan |
| 2. | hSMUG1 | hSMUG1 | uracil |
| 3. | hMBD4 | hMBD4 | U or T in U/TpG: 5meCpG |
| 4. | Mismatch-specific thymine/uracil | TDG | uracil (U: G), 3,N⁴-ethenocytosine DNA |
| | glycosylase | | (eC: G), T (T: G) |
| 5. | Methylpurine DNA glycosylase | MPG (ANPG, Aag) | 3-methyladenine, 7-methyladenine, |
| | | | 3-methylguanine, 7-methylguanine |
| | | | 8-oxoguanine, hypoxanthine, |
| | | | 1,N6-ethenoadenine, 1,N2-ethenoguanine |
| 6. | hNTH1 (human enodonuclease III | hNTH1 | thymine glycol, cytosine glycol, |
| | | | dihydrouracil, formamidopyrimidine urea |
| 7. | Adenine-specific mismatch DNA | hMYH | A from A: G; A: 8-oxoG; C: A |
| | glycosylase (human mutY homolog) | | 2-hydroxyadenine |
| . | 8-oxoguanine DNA glycosylase | hOGG1 | 2,5-amino-5-formamidopyrimidine |
| | | | 7,8-dihydro-8-oxoguanine |
| 9. | 8-oxo-GTPase/8-oxodGTPase | hMTH1 (NUDT1) | 8-oxo-GTP, 8-oxo-dGTP |
| | (Human MutT homolog) | | |
| 10. | dUTPase | hDUT | dUTP |
| 11. | AP endonuclease | APE1/APE2 | abasic site |
| 12. | Deoxyribose phosphate lyase | POLB | Incised abasic site |

Enzymes 1-8 are DNA glycosylases;
Enzymes 9 and 10 hydrolyze damaged or unnatural dNTPs, thereby preventing their incorporation into DNA during DNA synthesis.
Further details concerning mammalian DNA repair genes and activity can be found in Krokan et al. (2000) FEBS Letters 476: 73-77; and Wood et al. (2001) Science 291: 1284-1289, both are incorporated herein by reference.

The risk according to the present invention can be expressed in one of a plurality of ways. In one example the risk is expressed as a fold risk increase in developing cancer as is compared to a normal, apparently healthy, population. In another example, the risk is expressed in enzyme specific activity units. In another example, a linear or logarithmic risk scale is generated for either the “fold risk increase” or the “activity units” and the risk is expressed as a magnitude of the scale.

According to still further features in the described preferred embodiments determining the level of activity of the DNA repair/damage preventing enzyme is effected using a DNA substrate having at least one lesion therein.

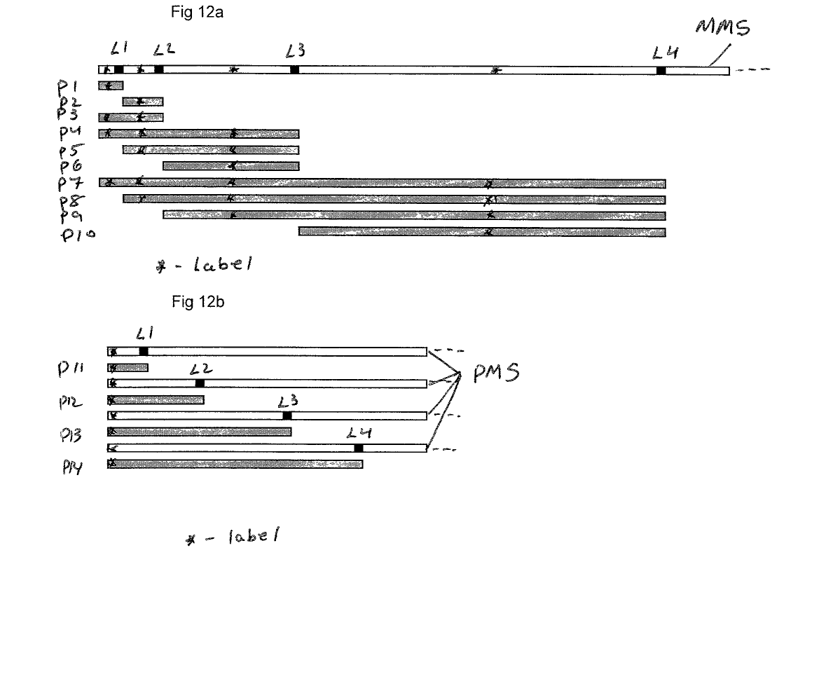

As is schematically exemplified by FIGS. 12a-b, a monomolecular (MMS, FIG. 12a) or plurimolecular (PMS, FIG. 12b ) universal substrate can also be generated and used while implementing the methods and kits of the present invention. Such a universal substrate is used according to the present invention to simultaneously determine the activity of more than a single DNA repair/damage preventing enzyme. Thus, a universal substrate of the invention includes at least two (four are shown in FIGS. 12a-b identified by L1-L4) different DNA lesions specifically recognized by at least two different DNA repair enzymes. Careful selection of the positions of the different DNA lesions along the universal substrate, can be used to ensure the generation of distinguishable (e.g., size distinguishable) reaction products (P1-P10 in FIG. 12a, P11-14 in FIG. 12b ), being indicative of the activity of the different DNA repair enzymes. In order to ensure accuracy, the lesions are selected to be unique to the activities tested. The length of the universal substrate, especially for a monomolecular substrate, which preferably includes labels along its length, is selected such that reciprocal reaction products are substantially longer than all of the reaction products to be analyzed (P1-P10 in FIG. 12a). End labeling can be used in the case of a plurimolecular substrate to circumvent this problem altogether. Thus, the length of a substrate according to the present invention, without limitation, can range between 10 base pairs and several hundreds base pairs.

A substrate of the invention can thus have at least one lesion of at least one type or at least one lesion of at least two types (universal substrate), the lesions preferably being positioned at predetermined site(s) in the DNA substrate. The lesion(s) can be of any type, including, but not limited to, uracil, 5-fluorouracil, 5-hydroxyuracil, isodialuric acid, alloxan, uracil or thymine in U/TpG:5meCpG, uracil (U:G), 3,N⁴-ethenocytosine, (eC:G), T (T:G), 3-methyladenine, 7-methyladenine, 3-methylguanine, 7-methylguanine, hypoxanthine, 1, N6-ethenoadenine, 1,N2-ethenoguanine, thymine glycol, cytosine glycol, dihydrouracil, formamidopyrimidine urea, adenine from A:G; A:8-oxoG; C:A, 2-hydroxyadenine, 2,5-amino-5-formamidopyrimidine, 7,8-dihydro-8-oxoguanine and abasic site..

A lesion can be introduced at a unique and defined location (site) in a DNA molecule using solid phase DNA synthesis, using in sequence the four conventional phosphoramidite building blocks used in the synthesis of oligodeoxynucleotides and additional at least one modified phosphoramidite building block, which when introduced into the DNA introduces a lesion therein, which lesion is recognizable by a DNA repair enzyme. In the alternative, a DNA molecule is exposed to a mutagenic agent (e.g., an oxidative agent or UV radiation) which forms one or more lesion of one or more types therein. Even when using this method, one can select a presubstrate which will result in a product (substrate of the invention) in which the lesions are non-randomly distributed, since the extent by which a specific lesion is formed in DNA is often dependent on the DNA sequence.

Other alternatives also exist. For example, one can oxidize a plasmid DNA with an oxidizing agent. This will form several lesions in the plasmid DNA. One can now use this plasmid DNA to assay a repair enzyme that acts on this DNA, without knowing precisely where the lesions are. The enzyme will produce a nick in the DNA, and this will convert the plasmid from the supercoiled closed form to the nicked (open circular) form. These two can be easily distinguished by gel electrophoresis or gradient centrifugation. In another example a piece of DNA is enzymatically synthesized in the presence of lesioned building blocks. Other alternatives are also known, such as chemical deamination, etc.

Thus, the substrate of the present invention can include at least two different lesions of at least two types, a single lesion, or at least two different lesions of a single type.

A cancer risk determination test according to the present invention is specifically advantageous for a subject which is known to be, or is about to be, exposed to environmental conditions associated with increased risk of developing cancer, such as smoking and occupational exposure to smoke, ionizing radiation and other carcinogens.

As is discussed hereinabove, the effectiveness of cancer therapy is due to its genotoxic effect affecting cancer cells more than normal cells. Thus, according to another aspect of the present invention there is provided a method of predicting the efficacy of a mutagenic anti-cancer treatment, such as chemotherapy and/or radiotherapy, in a subject. The method according to this aspect of the invention is effected by determining a level of activity of a DNA repair enzyme in a tissue of the subject, and, according to the level, predicting the efficacy of the mutagenic anti-cancer treatment in the subject.

Anti cancer therapy dosage can also be individually optimized in view of the teachings of the present invention. Thus, according to still another aspect of the present invention there is provided a method of selecting dosage of a mutagenic anti-cancer treatment, such as chemotherapy and/or radiotherapy, for treating a subject. The method according to this aspect of the invention is effected by determining a level of activity of a DNA repair/damage preventing enzyme in a tissue of the subject, and, according to the level, selecting dosage of the mutagenic anti-cancer treatment for treating the subject. In this case, the tissue is preferably a biopsy derived from the cancer itself.

According to an additional aspect of the present invention there is provided a kit for determining a level of activity of a DNA repair/damage preventing enzyme in a tissue of a subject. In its minimal configuration, the kit includes, a package including, contained in sealable containers, a DNA substrate having at least one lesion therein and a reaction buffer selected suitable for supporting DNA repair activity. Preferably, the kit also includes test tubes for separating lymphocytes. Preferably, the test tubes are prepackaged with an anti-coagulant, such as, but not limited to, heparin. Still preferably, the kit further includes a liquid having a specific gravity selected effective in separating lymphocytes from red blood cells via centrifugation, e.g., Ficoll contained in lymphocytes isolation tubes. Advantageously, the kit includes a solution having osmolarity selected effective in lysing red blood cells. In a preferred embodiment of the invention a protein extraction buffer is also included in the kit. Preferably, the kit further includes reagents for conducting protein determinations, e.g., reagents included in the BCA kit by Pierce. Still preferably, the kit includes a purified DNA repair enzyme, which serves as a control for such activity.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Materials and Experimental Methods

DNA substrates: The DNA substrate was prepared by annealing two complementary synthetic oligonucleotides, 32-bases long each. They were synthesized by the Synthesis Unit of the Biological Services Department at the Weizmann Institute of Science. The oligonucleotide containing 8-oxoG had the sequence 5′-CCGGTGCATGACACTGTOACCTATCCTCAGCG-3′ (SEQ ID NO: 1) (O=8-oxoG). The 8-oxoG phosphoramidite building block was purchased from Glen Research. It was ³²P-labeled using T4 polynucleotide kinase, and annealed to the oligonucleotide 5′-CGCTGAGGATAGGTCACAGTGTCATGCACCGG-3′ (SEQ ID NO:2). The radiolabeled duplex was purified by PAGE on a native 10% gel. Its concentration was determined by the PicoGreen dsDNA quantitation assay (Molecular Probes).

Blood samples: Large blood samples were obtained from the blood bank in the Sheba Medical Center. Samples of 10 ml peripheral blood were obtained from healthy donors or from cancer patients. Those were collected after obtaining permission from the Institutional Helsinki Committees.

Isolation of peripheral lymphocytes: The blood samples were processed 18-24 hours after collection. A 100 μl aliquot from each sample of whole blood was analyzed using a Cobas Micros (Roche Diagnostic System) blood counter. Ten ml PBS (Dulbecco's phosphate buffered saline, Sigma) were added to the remaining blood portion, and peripheral blood lymphocytes were isolated by density gradient centrifugation of the diluted whole blood on a polysucrose-sodium metrizoate medium in UNI-SEP tube (NOVAmed). Centrifugation was performed at 1,000 ×g for 30 minutes at 20° C.

Following centrifugation the lymphocyte band was removed and washed with PBS buffer. Elimination of red blood cells was done by lysis in 5 ml of 155 mM NH₄Cl; 0.01 M KHCO₃; 0.1 mM EDTA for 4 minutes at room-temperature. The lymphocytes were washed with PBS, and suspended in 1 ml PBS. The number of white blood cells in this suspension was determined using a Cobas Micros (Roche Diagnostic System) blood counter.

Samples containing 1-4×106⁶ cells were precipitated by centrifugation at 5,000 rpm, for 4 minutes at room temperature. The cells pellet was then resuspended to a concentration of 20,000 cells/μl in 50 mM Tris.HCl (pH 7.1), 1 mM EDTA, 0.5 mM DTT, 0.5 mM spermidine, 0.5 mM spermine, and a protease inhibitor cocktail (Sigma). The cells were incubated on ice for 30 minutes, after which they were frozen in liquid nitrogen. The frozen lymphocytes were stored at−80° C.

Preparation of a protein extract: The frozen lymphocytes were thawed at 30° C., after which their protein content was extracted with 220 mM KCl, for 30 minutes on ice. Cell debris was removed by centrifugation at 13,200 rpm for 15 minutes at 4° C. Glycerol was added to the protein extract to a final concentration of 10%, and the extract was frozen in liquid nitrogen. Protein concentration was determined by the BCA assay kit (Pierce) using bovine γ-globulin as a standard.

Standard analysis of OGG activity: The reaction mixture (20 μl) contained 50 mM TrisHCl (pH 7.1), 1 mM EDTA, 115 mM KCl, 20 μg bovine γ-globulin, 2 pmol PolydA.polydT, 0.5 pmol substrate and 8-12 μg protein extract. The reaction was carried out at 37° C. for 30 minutes, after which it was stopped by the addition of 15 mM EDTA, 0.2% SDS. The proteins were degraded by incubation with proteinase K (20 μg) for one hour at 37° C., after which they were treated with 80 mM NaOH for 30 minutes at 37° C. The denatured DNA products were analyzed by electrophoresis on a 15% polyacrylamide gel containing 8 M urea, in 89 mM Tris.borate, 2.5 mM EDTA pH 8.0, at 1,500 V for 2 hours at 45-50° C. The distribution of radiolabeled DNA products was visualized and quantified using a Fuji BAS 2500 phosphorimager. One unit of OGG activity is defined herein to cleave 1 fmol of DNA substrate in 1 hour at 37° C., under the standard reaction conditions described herein. In the following, OGGA is presented as specific activity, i.e., activity units/1 μg of total protein extract.

Statistical analysis: Mean OGGA values were compared using the student's two-tailed t-test, associations were calculated using Fisher's exact test, and Odds ratios were calculated from a 2×2 table. Adjusted Odds Ratios were calculated by logistic regression with the dependent variable being the condition (healthy/sick), and the independent variables being OGGA (dichotomous), age, and smoking status, as indicated. The software used was SAS version 6.12.

### Experimental Results

The OGG activity (OGGA) DNA repair test: Base excision repair (BER) is initiated by a DNA N-glycosylase, that releases the damaged or unusual base from DNA, generating an abasic site. The latter is then repaired by an AP endonuclease (APE/HAP1) and a deoxyribose phosphate lyase (part of DNA polymerase β), followed by re-synthesis of a short 1-3 nucleotides patch by DNA polymerase β, and ligation (Dianov, et al., 1992, Singhal, et al., 1995). A long patch pathway of BER was identified which requires also PCNA, and the FEN-1 flap endonuclease (Fortini, et al., 1998, Kim, et al, 1998). It was reported that 8-oxoG can be repaired in cell extracts also by nucleotide excision repair (Reardon, et al., 1997), however, the in vivo significance of this finding is not clear (Runger, et al., 1995). In addition, it was reported that there is transcription-coupled repair of 8-oxoG, and that it required the XPG, TFIIH and CSB (Le Page, et al., 2000), and the BRCA1 and BRCA2 proteins (Le Page, et al., 2000).

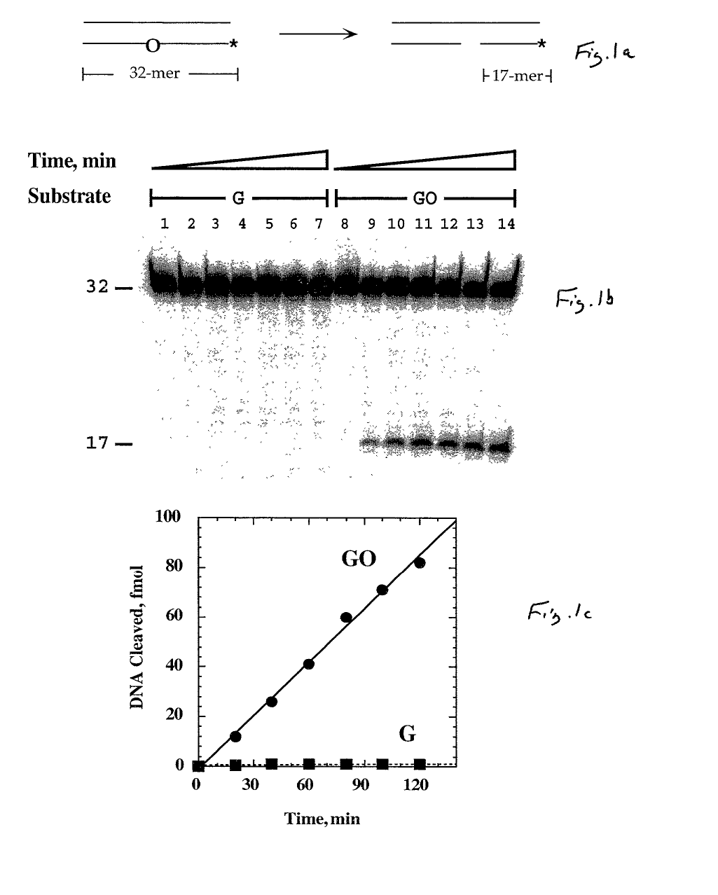

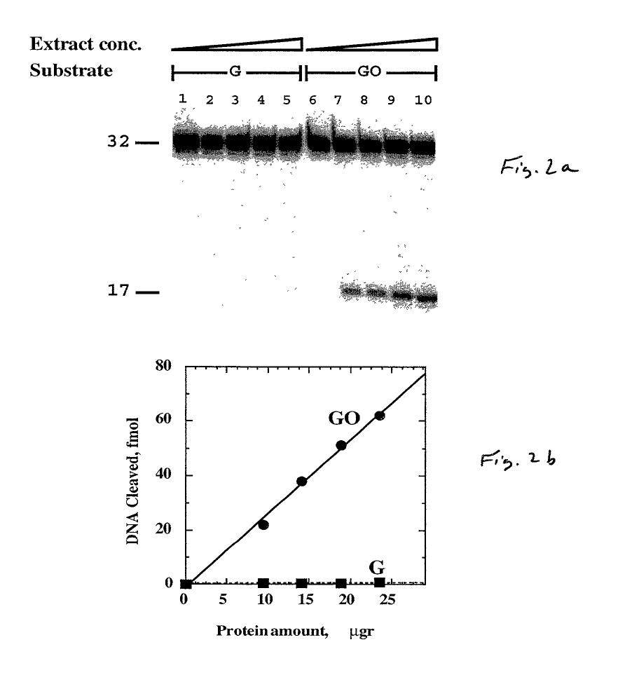

While reducing the present invention to practice, an assay was developed for OGG activity (OGGA), using as substrate a ³²P end-labeled synthetic oligonucleotide, 32-nucleotides long, carrying a site-specific 8-oxoG. The source of OGGA was a protein extract prepared from human peripheral lymphocytes, obtained by Ficoll fractionation from 10 ml blood samples. A protein extract is prepared from the lymphocytes by freeze-thawing, followed by salt extraction. The removal of 8-oxoG from the oligonucleotide, by the OGGA in the extract, generates an abasic site, which is rapidly incised either by the AP lyase activity of the enzyme, or by AP endonucleases present in the extract. Subsequent alkali treatment, which breaks abasic sites, did not affect DNA cleavage, but it is added in order to ensure that only glycosylase activity is assayed. Analysis by urea-PAGE followed by phosphorimaging was used to quantify the extent of nicking, indicated by the formation of a shorter radiolabeled DNA fragment, 17 nucleotides long (FIG. 1a). OGGA is measured as specific activity, i.e., units of enzyme activity/μg of total protein extract. One unit of activity cleaves 1 fmol of DNA substrate in 1 hour at 37° C., under standard reaction conditions.

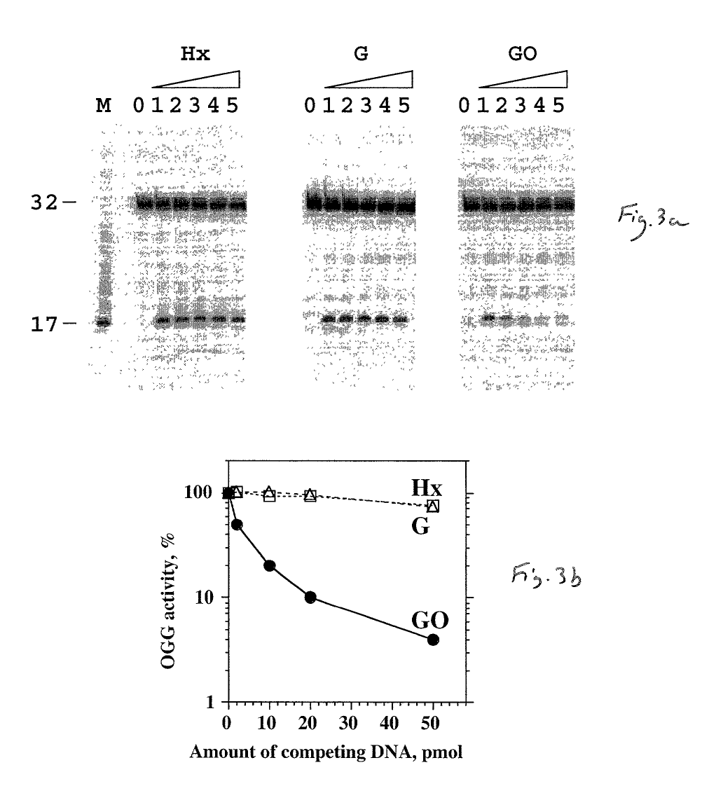

FIGS. 1b-c and 2a-b show a time course and a titration of OGGA, respectively, in lymphocyte extracts. The activity was dependent on the presence of 8-oxoG in the DNA substrate. No activity was observed when the DNA contained a G instead of the 8-oxoG. This activity is likely to be, at least in part, due to the OGGl enzyme, which was recently shown to be responsible for most (but not all) of OGG activity in extracts prepared from human cells (Monden, et al., 1999). Recently, the existence of OGG2, a second OGG enzyme was reported. However, its activity was much lower than OGGl in whole cell extracts (Hazra, et al., 1998). In addition to OGG, APNG (alkylpurine DNA N-glycosylase), also termed Aag (alkyladenine DNA glycosylase), or MPG (N-methylpurine glycosylase), was reported to act on 8-oxoG in one study (Bessho, et al., 1993), but not in another (Hang, et al., 1997). In vivo this protein has no significant role in removing 8-oxoG from DNA, at least in mice (Engelward, et al., 1997, Hang, et al, 1997). In order to establish whether this enzyme is involved in the removal of 8-oxoG from DNA by lymphocyte extracts, competition experiments were performed with substrates containing site-specific hypoxanthine (a substrate of APNG but not for OGGl; (see, Engelward, et al., 1997, Hang, et al., 1997)). As can be seen in FIG. 3, this substrate showed no inhibition of the activity of the extract on 8-oxoG-containing DNA, suggesting that APNG is not involved. A control with a G instead of 8-oxoG showed no inhibition, and a control 8-oxoG-DNA did show inhibition, as expected. These competition experiments are an indication of the specificity of OGGA to 8-oxoG.

Reproducibility experiments showed that the assay is accurate and highly reproducible, with a coefficient of variance of 10%. An example of a reproducibility experiment is shown in Table 2.

TABLE 2
Reproducibility of the OGGA test

A
Blood sample:

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

OGGA (units/μg protein):

| | | | | | | | | | | | |
|---|---|---|---|---|---|---|---|---|---|---|---|
| 6.8 | 6.9 | 6.4 | 6.5 | 6.7 | 7.4 | 6.9 | 6.5 | 6.6 | 6.7 | 5.9 | 6.9 |

| | |
|---|---|
| Average OGGA: | 6.7 |
| Standard variation: | 0.4 |
| Coefficient of variance: | 6% |

Twelve blood samples from a healthy donor (donor No. 54),
10 ml each, were processed and assayed for OGGA. One unit of
OGG activity incises 1 fmol GO-containing substrate in 60
minutes at 37° C. under standard assay conditions.
B
Blood Sample:

| | | | | | | | | |
|---|---|---|---|---|---|---|---|---|
| 1 | 2 | 3 | 4 | 5 | 6 | Ave | SD | CV |

OGGA (units/μg protein)

| | | | | | | | | | |
|---|---|---|---|---|---|---|---|---|---|
| Experiment 1: | 6.7 | 7.2 | 6.7 | 6.8 | 7.1 | 6.7 | 6.9 | 0.2 | 3% |
| Experiment 2: | 7.9 | 6.9 | 7.8 | 8.2 | 8.1 | 7.8 | 7.8 | 0.5 | 6% |
| Experiment 3: | 6.9 | 7.0 | 7.1 | 7.3 | 7.9 | 7.2 | 7.2 | 0.4 | 5% |

| | |
|---|---|
| Overall average OGGA: | 7.1 |
| Standard deviation (SD): | 0.5 |
| Coefficient of variance (CV): | 7% |

Six blood samples from a healthy donor (donor No. 50),
10 ml each, were processed to prepare protein extracts.
The table shows the results of three independent assays
performed with these assays on three different days.

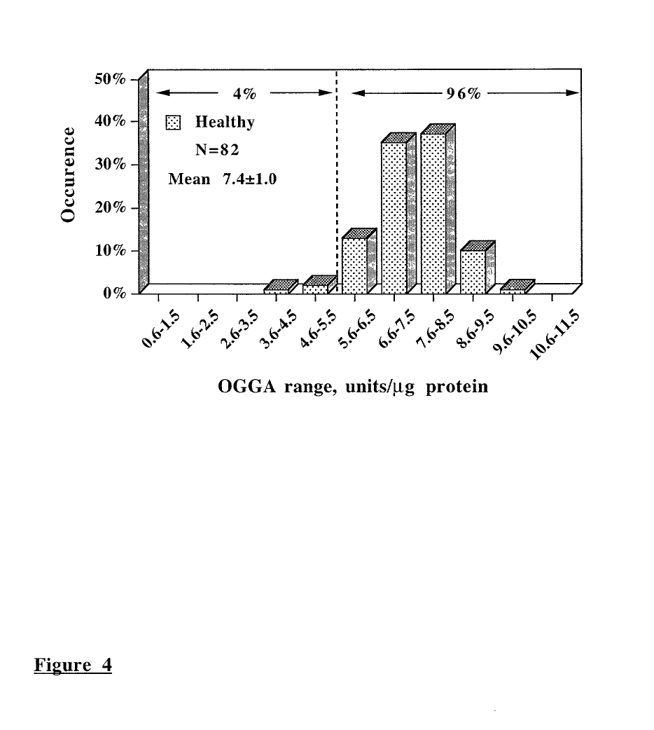

OGGA index in healthy individuals: The OGGA test was performed on blood samples from 82 healthy individuals, and the distribution is shown in FIG. 4. The mean OGGA index was 7.41±1.02 units/μg protein (Table 3).

TABLE 3
OGGA values in healthy individuals

| Factor | No. | Mean OGGA ± SD* | P** |
|---|---|---|---|
| All | 82 | 7.41 ± 1.02 | |
| Age | | | |
| 20-49 | 35 | 7.62 ± 0.87 | 0.107 |
| 50-80 | 47 | 7.25 ± 1.11 | |
| Sex | | | |
| Male | 35 | 7.71 ± 0.95 | 0.019 |
| Female | 47 | 7.19 ± 1.03 | |
| Smoking | | | |
| Never | 45 | 7.39 ± 1.06 | 0.93 |
| Current | 34 | 7.38 ± 1.00 | |

*SD, standard deviation.
**P values were obtained from student's two-tailed t-test analysis between the two sub-groups in each category.

The range of OGGA was 3.6-10.1 units/μg protein, representing a 2.8-fold range of OGG activity. If the lowest case is eliminated (it might represent a predisposition; see below), the range is 5.0-10.1 units/μg protein, only 2-fold variation. This is a rather narrow distribution of activity, significantly narrower than previously reported (Asami, et al., 1996).

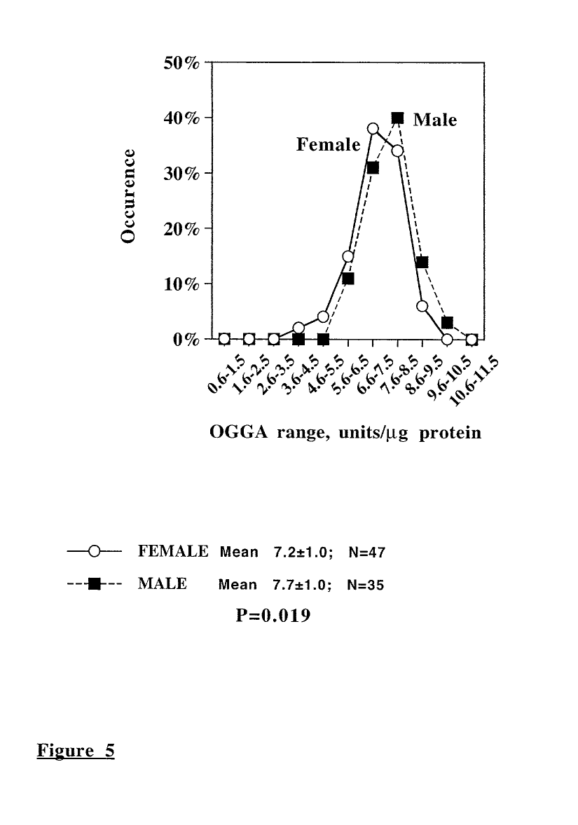

Analysis of the gender effect revealed a slightly lower activity in females (47 individuals; 7.19±1.03 units/μg protein) as compared to males (35 individuals; 7.71±0.95 units/μg protein; FIG. 5; Table 3). This difference was statistically significant (P=0.019).

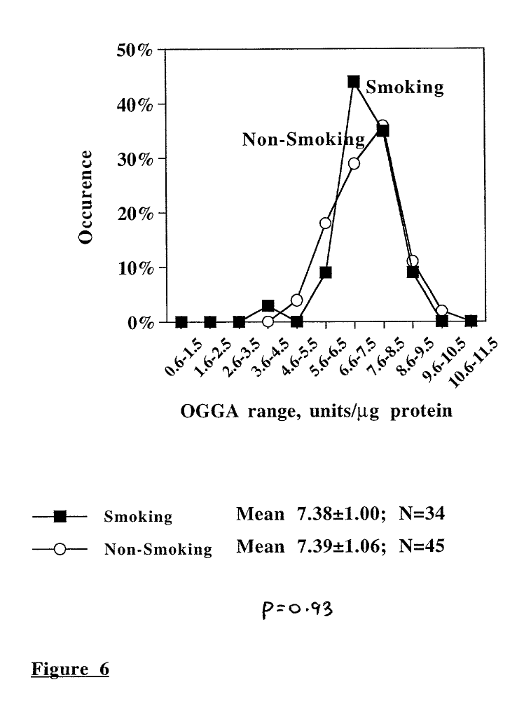

A comparison of the OGGA test in smokers (34 individuals) yielded a mean value of 7.38±1.00 units/μg protein, essentially identical to the OGGA index of 7.39±1.06 units/μg protein obtained in non-smokers (45 individuals). This indicates that smoking does not affect the OGGA index in peripheral blood lymphocytes (FIG. 6; Table 3). This result differs from the result obtained by Asami et al. (1996), who reported that 8-oxoG repair activity was increased 1.6-fold in smokers.

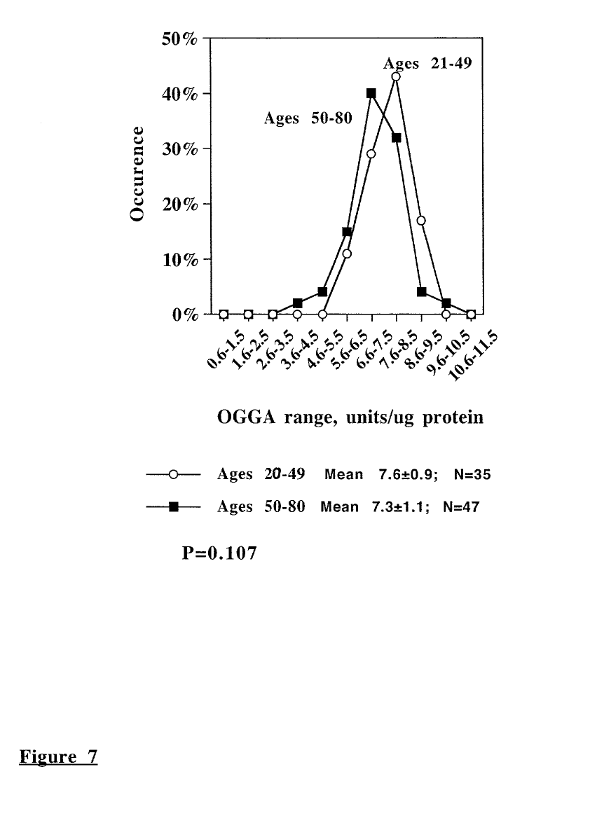

The mean OGGA index in healthy individuals of ages 20-49 (7.62±0.87; N=35) was similar to healthy individuals of ages 50-80 (7.25±1.11; N=47), the difference being statistically not significant (P=0.107; FIG. 7; Table 3). Taken together these results indicate little or no variation of the OGGA index with age, smoking status and gender.

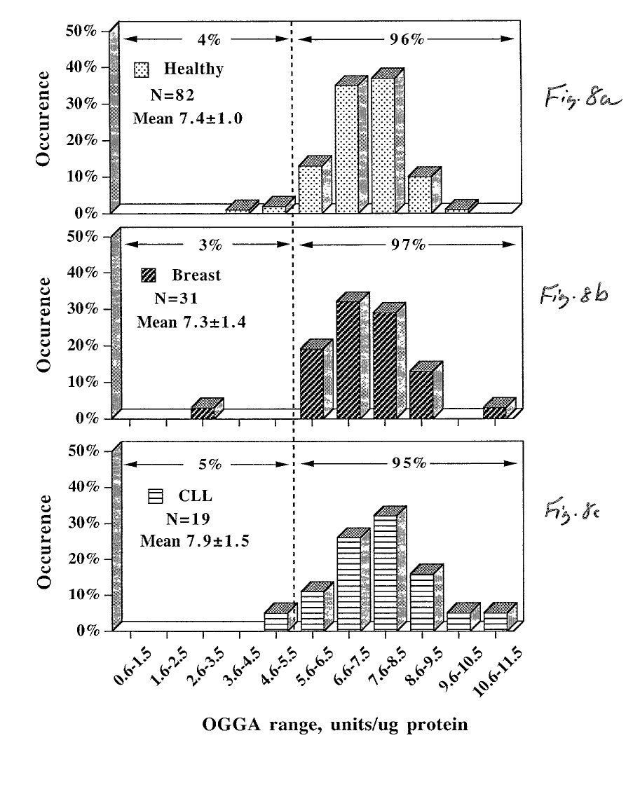

OGGA is not reduced in patients with breast cancer or chronic lymphocytic leukemia (CLL): The OGGA test was performed on blood samples from 31 breast cancer patients and 19 CLL patients. As can be seen in FIGS. 8a-c, the mean OGGA index was 7.28±1.39 units/μg protein in breast cancer patients and 7.90±1.52 units/μg protein in CLL patients, similar to that of healthy individuals. Also the distribution of OGGA values was similar (FIGS. 8a-c; Table 4). These results indicate that OGGA is not a risk factor in breast cancer or in CLL.

TABLE 4
Mean OGGA values in cancer patients

| Healthy/Disease | No. | Mean OGGA ± SD* | P** |
|---|---|---|---|
| Healthy | 82 | 7.41 ± 1.02 | |
| Lung cancer | 54 | 6.19 ± 1.63 | 0.0001 |
| (NSCLC) | | | |
| Breast cancer | 31 | 7.28 ± 1.39 | 0.720 |
| Lymphoma | 18 | 6.16 ± 1.84 | 0.0117 |
| CLL | 19 | 7.90 ± 1.52 | 0.20 |
| Colorectal cancer | 16 | 7.52 ± 1.76 | 0.813 |

*SD, standard deviation.
**P values were obtained from student's two-tailed t-test analysis between the indicated disease and the control healthy group.

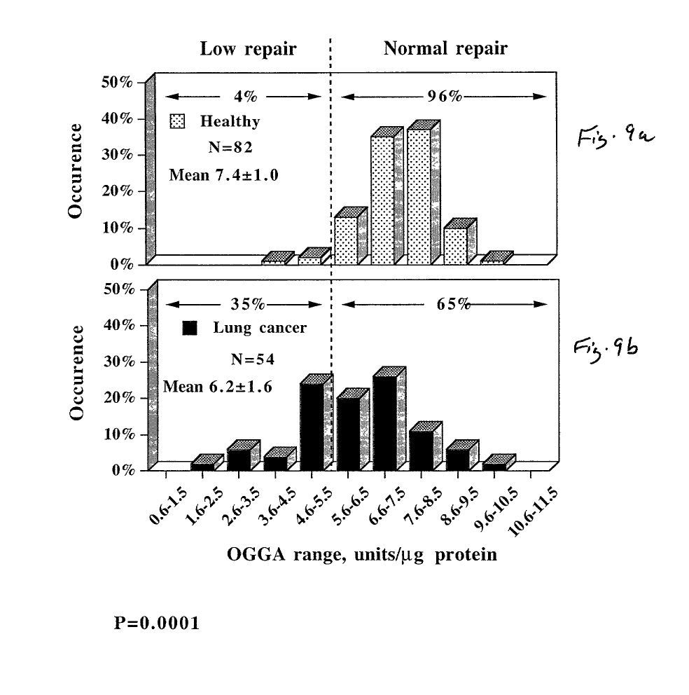

OGGA is reduced in patients with lung cancer: The OGGA test was performed with blood samples from 54 newly diagnosed lung cancer patients who did not undergo therapy, and before undergoing surgery. All cases were diagnosed for NSCLC. As can be seen in FIGS. 9a-b and Table 4, the mean OGGA was 6.19±1.63 units/μg protein, significantly lower than in healthy individuals (P=0.0001). More importantly, OGGA index was remarkably lower in a substantial fraction of patients. ‘Low’ OGGA values are herein defined as values of 5.5 units/μg protein and lower, whereas values above 5.5 units/μg protein are considered ‘Normal’. Among healthy individuals 4% had Low OGGA, whereas 35% of all lung cancer patients had Low OGGA. This includes values 2-3 fold lower than the mean OGGA values of healthy people. Only 1 out of 82 healthy individuals had such a very low OGG value. This individual is a smoker with a family history of lung cancer.

Low OGG is a strong risk factor in lung cancer: Analysis of Normal and Low repair in healthy individuals and in lung cancer patients using Fisher's Exact test (Table 5) yielded an adjusted Odds Ratio of 13.89 (95% confidence interval, 2.53-76.9). In other words, after adjustment for age and smoking status, lung cancer patients were 14 times more likely than the healthy controls to have a Low OGGA. When only smokers were considered (34 healthy; 41 lung cancer patients), the Odds Ratio was 25.86 (95% CI, 3.22-207.3). These results indicate that Low OGGA is a strong risk factor in lung cancer (Table 5). This analysis was also performed with non-smokers, resulting in an insignificant association between Low OGGA and lung cancer in non-smokers (Table 5).

TABLE 5
Association of Low OGGA and lung cancer

| Factor | Cases | Controls | Crude OR (CI*) | Adjusted** OR (CI) |
|---|---|---|---|---|
| OGGA | | | | |
| (total population) | | | | |
| Normal > 5.5 | 35 | 79 | 1.00 | 1.00 |
| Low ≦ 5.5 | 19 | 3 | 14.30 (3.97-51.48) | 13.89 (2.53-76.9) |
| | | | P = 0.0000 | |
| Smoking status | | | | |
| (total population) | | | | |
| Non-smokers | 10 | 45 | 1.00 | |
| Smokers | 41 | 34 | 5.43 (2.39-12.35) | |
| | | | P = 0.0000 | |
| OGGA | | | | |
| (among smokers) | | | | |
| Normal > 5.5 | 23 | 33 | 1.00 | |
| Low ≦ 5.5 | 18 | 1 | 25.83 (3.22-207.3) | |
| | | | P = 0.000 | |
| OGGA | | | | |
| (among non-smokers | | | | |
| Normal > 5.5 | 9 | 43 | 1.00 | |
| Low ≦ 5.5 | 1 | 2 | 2.39 (0.20-29.27) | |
| | | | P = 0.459 | |

*CI, 95% confidence interval.
**Adjusted for age and smoking status.

Based on the data presented herein it is possible to evaluate the increased risk in lung cancer associated with Low OGGA. This was done according to Vandenbroucke, et al. (1994). Smokers with Low OGGA have a 25.8-fold higher risk for developing lung cancer compared to smokers with Normal OGGA (Table 6). For non-smokers the increased risk is marginal (2.39-fold; Table 6). This means that low OGGA sensitizes individuals to smoking-induced lung cancer.

TABLE 6
Low OGGA Strongly Increases the Risk of Lung Cancer in Smokers

Ratio of Cases/controls

| | Normal OGGA | Low OGGA |
|---|---|---|
| Non-smokers | 0.209 | 0.5 |
| Smokers | 0.697 | 18 |

Lung cancer risk for smokers who have Low OGGA, is 25.8-fold higher than for smokers who have Normal OGGA. Risk was calculated according to (Vandenbroucke, et al., 1994) as follows: For smokers: 18/0.697 = 25.8; For non-smokers the risk is 0.5/0.209 = 2.39.

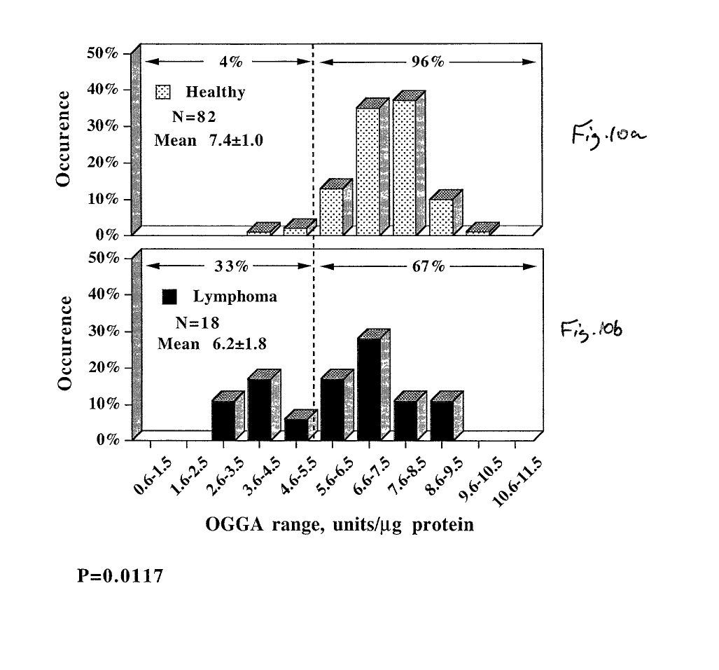

Low OGG activity is a risk factor in lymphoma: Analysis of 19 lymphoma patients showed a clear shift to lower OGG DNA repair values (FIG. 10a-b; Table 4): The mean OGGA index was 6.16±1.84 units/μg protein, significantly lower than in healthy individuals (P=0.0117). Analysis of Normal and Low repair in healthy individuals and in lymphoma patients using Fisher's Exact test yielded an adjusted Odds Ratio of 12.66 (95% CI, 2.66-58.82). This means that after adjustment for age, lymphoma patients were 13 times more likely than the healthy controls to have a Low OGGA. This indicates that Low OGGA is a risk factor in lymphoma (Table

TABLE 7
Association of Low OGGA and lymphoma

| Factor | Cases | Controls | Crude OR (CI*) | Adjusted** OR (CI) |
|---|---|---|---|---|
| OGGA | | | | |
| Normal > 5.5 | 12 | 79 | 1.00 | 1.00 |
| Low ≦ 5.5 | 6 | 3 | 13.27 (2.9-59.79) | 12.66 (2.66-58.82) |
| | | | P = 0.004 | |

*CI, 95% confidence interval.
**Adjusted for age.

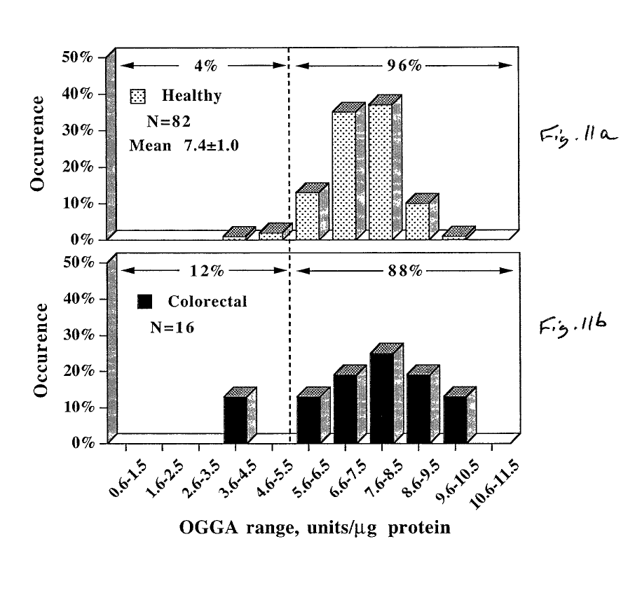

OGG activity seems to be reduced in colorectal cancer patients: An analysis was performed with 16 colorectal cancer patients (FIGS. 11a-b). Two of the patients exhibited low OGG (12%). This data is indicative that low OGG is a risk factor in colorectal cancer.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### References Cited:

Aburatani, H., Y. Hippo, T. Ishida, R. Takashima, C. Matsuba, T. Kodama, M. Takao, A. Yasui, K. Yamamoto and M. Asano. (1997) Cloning and characterization of mammalian 8-hydroxyguanine-specific DNA glycosylase/apurinic, apyrimidinic lyase, a functional mutM homologue, Cancer Res., 57, 2151-2156.

Arai, K., K. Morishita, K. Shinmura, T. Kohno, S. R. Kim, T. Nohmi, M. Taniwaki, S. Ohwada and J. Yokota. (1997) Cloning of a human homolog of the yeast OGGI gene that is involved in the repair of oxidative damage, Oncogene, 14, 2857-2861.

Asami, S., T. Hirano, R. Yamaguchi, Y. Tomioka, H. Itoh and H. Kasai (1996) Increase of a type of oxidative DNA damage, 8-hydroxyguanine, and its repair activity in human leukocytes by cigarette smoking, Cancer Res., 56, 2546-2549.

Asami, S., H. Manabe, J. Miyake, Y. Tsurudome, T. Hirano, R. Yamaguchi, H. Itoh and H. Kasai. (1997) Cigarette smoking induces an increase in oxidative DNA damage, 8-hydroxydeoxyguanosine, in a central site of the human lung, Carcinogenesis, 18, 1763-1766.

Athas, W. F., M. A. Hedayati, G. M. Matanoski, E. R. Farmer and L. Grossman. (1991) Development and field-test validation of an assay for DNA repair in circulating human lymphocytes, Cancer Res., 51, 5786-5793.

Audebert, M., S. Chevillard, C. Levalois, G. Gyapay, A. Vieillefond, J. Klijanienko, P. Vielh, A. K. El Naggar, S. Oudard, S. Boiteux and J. P. Radicella. (2000) Alterations of the DNA repair gene OGGl in human clear cell carcinomas of the kidney, Cancer Res., 60, 4740-4744.

Bessho, T., R. Roy, K. Yamamoto, H. Kasai, S. Nishimura, K. Tano and S. Mitra. (1993) Repair of 8-hydroxyguanine in DNA by mammalian N-methylpurine-DNA glycosylase, Proc. Natl. Acad. Sci. USA, 90, 8901-8904.

Bishop, J. M. (1995) Cancer: The rise of the genetic paradigm, Genes & Dev., 9, 1309-1315.

Bjoras, M., L. Luna, B. Johnsen, E. Hoff, T. Haug, T. Rognes and E. Seeberg. (1997) Opposite base-dependent reactions of a human base excision repair enzyme on DNA containing 7,8-dihydro-8-oxoguanine and abasic sites, EMBO J., 16, 6314-6322.

Chevillard, S., J. P. Radicella, C. Levalois, J. Lebeau, M. F. Poupon, S. Oudard, B. Dutrillaux and S. Boiteux. (1998) Mutations in OGGl, a gene involved in the repair of oxidative DNA damage, are found in human lung and kidney tumours, Oncogene, 16, 3083-3086.

Connor, F., D. Bertwistle, P. J. Mee, G. M. Ross, S. Swift, E. Grigorieva, V. L. J. Tybulewicz and A. Ashworth. (1997) Tumorigenesis and a DNA repair defect in mice with a truncating Brca2 mutation, Nature Genet, 17, 423-430.

Dianov, G., A. Price and T. Lindahl. (1992) Generation of single-nucleotide repair patches following excision of uracil residues from DNA, Mol. Cell Biol., 12, 1605-1612.

Echols, H. and M. F. Goodman. (1991) Fidelity mechanisms in DNA replication, Annu. Rev. Biochem., 60, 477-511.

Engelward, B. P., G. Weeda, M. D. Wyatt, J. L. Broekhof, J. de Wit, I. Donker, J. M. Allan, B. Gold, J. H. Hoeijmakers and L. D. Samson. (1997) Base excision repair deficient mice lacking the Aag alkyladenine DNA glycosylase, Proc. Natl. Acad. Sci. USA, 94, 13087-13092.

Fishel, R., M. K. Lescoe, M. R. Rao, N. G. Copeland, N. A. Jenkins, J. Garber, M. Kane and R. Kolodner. (1993) The human mutator gene homolog MSH2 and its association with hereditary nonpolyposis colon cancer, Cell, 75, 1027-38.

Fortini, P., B. Pascucci, E. Parlanti, R. W. Sobol, S. H. Wilson and E. Dogliotti. (1998) Different DNA polymerases are involved in the short-and long-patch base excision repair in mammalian cells, Biochemistry, 37, 3575-3580.

Friedberg, E. C., G. C. Walker and W. Siede (1995) DNA repair and mutagenesis, ASM Press, Washington, D. C.

Gajewski, E., G. Rao, Z. Nackerdien and M. Dizdaroglu. (1990) Modification of DNA bases in mammalian chromatin by radiation-generated free radicals, Biochemistry, 29, 7876-7882.

Gowen, L. C., A. V. Avrutskaya, A. M. Latour, B. H. Koller and S. A. Leadon. (1998) BRCA1 required for transcription-coupled repair of oxidative DNA damage, Science, 281, 1009-1012.

Hanawalt, P. C. (1994) Transcription-coupled repair and human disease, Science, 266, 1957-1958.

Hang, B., B. Singer, G. P. Margison and R. H. Elder. (1997) Targeted deletion of alkylpurine-DNA-N-glycosylase in mice eliminates repair of 1, N6-ethenoadenine and hypoxanthine but not 3, N4-ethenocytosine or 8-oxoguanine, Proc. Natl. Acad. Sci. USA, 94, 12869-12874.

Hazra, T. K., T. Izumi, L. Maidt, R. A. Floyd and S. Mitra. (1998) The presence of two distinct 8-oxoguanine repair enzymes in human cells: their potential complementary roles in preventing mutation., Nucleic Acids Res., 26, 5116-5122.

Helzlsouer, K. J., E. L. Harris, R. Parshad, H. R. Perry, F. M. Proce and K. K. Sanford. (1996) DNA repair proficiency: Potential susceptibility factor for breast cancer, J. Natl. Cancer. Inst., 88, 754-755.

Hemandez-Boussard, T., P. Rodriguez-Tome, R. Montesano and P. Hainaut, P. (1999) IARC p53 mutation database: a relational database to compile and analyze p53 mutations in human tumors and cell lines. International Agency for Research on Cancer, Hum. Mutat., 14, 1-8.

Hutchinson, F. (1985) Chemical changes induced in DNA by ionizing radiation, Prog. Nucleic Acid Res. Mol. Biol., 32, 115-154.

Hyun, J. W., J. Y. Choi, H. H. Zeng, Y. S. Lee, H. S. Kim, S. H. Yoon and M. H. Chung. (2000) Leukemic cell line, KG-1 has a functional loss of hOGG1 enzyme due to a point mutation and 8-hydroxydeoxyguanosine can kill KG-1, Oncogene, 19, 4476-4479.

Ishida, T., Y. Hippo, Y. Nakahori, I. Matsushita, T. Kodama, S. Nishimura and H. Aburatani. (1999) Structure and chromosome location of human OGGI, Cytogenet. Cell Genet., 85, 232-236.

Jyothish, B., R. Ankathil, B. Chandini, B. Vinodkumar, G. Sunil Nayar, D. Dinesh Roy, J. Madhavan and M. Krishnan Nair. (1998) DNA repair proficiency: a potential marker for identification of high risk members in breast cancer families, Cancer Lett., 124, 9-13.

Kim, K., S. Biade and Y. Matsumoto. (1998) Involvement of flap endonuclease 1 in base excision DNA repair, J. Biol. Chem., 273, 8842-8848.

Klungland, A., I. Rosewell, S. Hollenbach, E. Larsen, G. Daly, B. Epe, E. Seeberg, T. Lindahl and D. E. Barnes. (1999) Accumulation of premutagenic DNA lesions in mice defective in removal of oxidative base damage, Proc. Natl. Acad. Sci. USA, 96, 13300-13305.

Laval, F. (1994) Expression of the E. coli fpg gene in mammalian cells reduces mutagenicity of g rays, Nucleic Acids Res., 22, 4943-4946.

Le Page, F., E. E. Kwoh, A. Avrutskaya, A. Gentil, S. A. Leadon, A. Sarasin and P. K. Cooper. (2000) Transcription-coupled repair of 8-oxoguanine: requirement for XPG, TFIIH, and CSB and implications for Cockayne syndrome, Cell, 101, 159-171.

Le Page, F., V. Randrianarison, D. Marot, J. Cabannes, M. Perricaudet, J. Feunteun and A. Sarasin. (2000) BRCA1 and BRCA2 are necessary for the transcription-coupled repair of the oxidative 8-oxoguanine lesion in human cells, Cancer Res., 60, 5548-5552.

Leach, F. S., N. C. Nicolaides, N. Papadopoulos, B. Liu, J. Jen, R. Parsons, P. Peltomaki, P. Sistonen, L. A. Aaltonen, M. Nystromlahti, X. Y. Guan, J. Zhang, P. S. Meltzer, J. W. Yu, F. T. Kao, D. J. Chen, K. M. Cerosaletti, R. E. K. Fournier, S. Todd, T. Lewis, R. J. Leach, S. L. Naylor, J. Weissenbach, J. P. Mecklin, H. Jarvinen, G. M. Petersen, S. R. Hamilton, J. Green, J. Jass, P. Watson, H. T. Lynch, J. M. Trent, J. M. Trent, A. de la Chapelle, K. W. Kinzler and B. Vogelstein. (1993) Mutations of a mutS homolog in hereditary nonpolyposis colorectal cancer, Cell, 75, 1215-1225.

Leanderson, P. and C. Tagesson. (1992) Cigarette smoke-induced DNA damage in cultured human lung cells: role of hydroxyl radicals and endonuclease activation, Che. Biol. Interact., 81, 197-208.

Livneh, Z., O. Cohen-Fix, R. Skaliter and T. Elizur. (1993) Replication of damaged DNA and the molecular mechanism of ultraviolet light mutagenesis, CRC Crit. Rev. Biochem. Mol. Biol., 28, 465-513.

Lu, R., H. M. Nash and G. L. Verdine. (1997) A mammalian DNA repair enzyme that excises oxidatively damaged guanines maps to a locus frequently lost in lung cancer., Curr. Biol., 7, 397-407.

Maki, H. and M. Sekiguchi. (1992) MutT protein specifically hydrolyses a potent mutagenic substrate for DNA synthesis, Nature (London), 355, 273-275.

Minowa, O., T. Arai, M. Hirano, Y. Monden, S. Nakai, M. Fukuda, M. Itoh, H. Takano, Y. Hippou, H. Aburatani, K. Masumura, T. Nohmi, S. Nishimura and T. Noda. (2000) Mmh/Oggl gene inactivation results in accumulation of 8-hydroxyguanine in mice, Proc. Natl. Acad. Sci. USA, 97, 4156-4161.

Modrich, P. (1994) Mismatch repair, genetic stability, and cancer, Science, 266, 1959-1960.

Monden, Y., T. Arai, M. Asano, E. Ohtsuka, H. Aburatani and S. Nishimura. (1999) Human MMH (OGGl) Type la Protein Is a Major Enzyme for Repair of 8-Hydroxyguanine Lesions in Human Cells, Biochem. Biophys. Res. Commun., 258, 605-610.

Parshad, R., F. M. Price, V. A. Bohr, K. H. Cowans, J. A. Zujewski and K. K. Sanford. (1996) Deficient DNA repair capacity, a predisposing factor in breast cancer, Brit. J. Cancer, 74, 1-5.

Parsons, R., G. M. Li, M. J. Longley, W. H. Fang, N. Papadopoulos, J. Jen, l. C. A. de, K. W. Kinzler, B. Vogelstein and P. Modrich. (1993) Hypermutability and mismatch repair deficiency in RER+tumor cells, Cell, 75, 1227-36.

Patel, K. J., V. P. C. C. Yu, H. Lee, A. Corcoran, F. C. Thistlethwaite, M. J. Evans, W. H. Colledge, L. S. Friedman, B. A. J. Ponder and A. R. Venkitaraman. (1998) Involvement of Brca2 in DNA Repair, Molecular Cell, 1, 347-357.

Patel, R. K., A. H. Trivedi, D. C. Arora, J. M. Bhatavdekar and D. D. Patel. (1997) DNA repair proficiency in breast cancer patients and their first-degree relatives, Int. J. Cancer, 73, 20-24.

Pavlov, Y. I., D. T. Minnick, S. Izuta and T. A. Kunkel. (1994) DNA replication fidelity with 8-oxodeoxyguanosine triphosphate, Biochemistry, 33, 4695-4701.

Radicella, J. P., C. Dherin, C. Desmaze, M. S. Fox and S. Boiteux. (1997) Cloning and characterization of hOGGl, a human homolog of the OGGl gene of Saccharomyces cerevisiae, Proc. Nati. Acad. Sci. USA, 94, 8010-8015.

Reardon, J. T., T. Bessho, H. C. Kung, P. H. Bolton and A. Sancar. (1997) In vitro repair of oxidative DNA damage by human nucleotide excision repair system: possible explanation for neurodegeneration in xeroderma pigmentosum patients, Proc. Natl. Acad. Sci. USA, 94, 9463-9468.

Roldan-Arjona, T., Y. F. Wei, K. C. Carter, A. Klungland, C. Anselmino, R. P. Wang, M. Augustus and T. Lindahl. (1997) Molecular cloning and functional expression of a human cDNA encoding the antimutator enzyme 8-hydroxyguanine DNA glycosylase, Proc. Natl. Acad. Sci. USA, 94, 8016-8020.

Rosenquist, T. A., D. O. Zharkov and A. P. Grollman. (1997) Cloning and characterization of a mammalian 8-oxoguanine DNA glycosylase, Proc. Natl. Acad. Sci. USA, 94, 7429-7434.

Runger, T. M., B. Epe and K. Moller. (1995) Repair of ultraviolet B and singlet oxygen-induced DNA damage in xeroderma pigmentosum cells, J. Invest. Dermatol., 104, 68-73.

Sagher, D., T. Karrison, J. L. Schwartz, R. Larson, P. Meier and B. Strauss. (1988) Low O6-alkylguanine DNA alkyltransferase activity in the peripheral blood lymphocytes of patients with therapy-related acute nonlymphocytic leukemia, Cancer Res., 48, 3084-3089.

Sancar, A. (1994) Mechanisms of DNA repair, Science, 266, 1954-1956.

Savitsky, K., A. Bar-Shira, S. Gilad, G. Rotman, Y. Ziv, L. Vanagaite, D. A. Tagle, S. Smith, T. Uziel, S. Sfez, M. Ashkenazi, I. Pecker, M. Frydman, R. Harnik, S. R. Patanjali, A. Simmons, G. A. Clines, A. Sartiel, R. A. Gatti, L. Chessa, O. Sanal, M. Lavin, N. G. J. Jaspers, A. M. R. Taylor, C. F. Arlett, T. Miki, S. M. Weissman, M. Lovett, F. S. Collins and Y. Shiloh. (1995) A single Ataxia Telangiectasia gene with a product similar to PI-3 kinase, Science, 268, 1749-1753.

Scully, R., J. Chen, A. Plug, Y. Xiao, D. Weaver, J. Feunteun, T. Ashley and D. M. Livingston. (1997) Association of BRCA1 with Rad51 in Mitotic and Meiotic cells, Cell, 88, 265-275.

Sharan, S. K., M. Morimatsu, U. Albrecht, D. S. Lim, E. Regel, C. Dinh, A. Sands, G. Eichele, P. Hasty and A. Bradley. (1997) Embryonic lethality and radiation hypersensitivity mediated by Rad51 in mice lacking Brca2, Nature, 386, 804-810.

Shibutani, S., M. Takeshita and A. P. Grollman. (1991) Insertion of specific bases during DNA synthesis past the oxidation-damaged base 8-oxodG, Nature, 349, 431-434.

Shinmura, K., T. Kohno, H. Kasai, K. Koda, H. Sugimura and J. Yokota. (1998) Infrequent mutations of the hOGGl gene, that is involved in the excision of 8-hydroxyguanine in damaged DNA, in human gastric cancer, Jpn. J. Cancer Res., 89, 825-828.

Singhal, R. K., R. Prasad and S. H. Wilson. (1995) DNA polymerase beta conducts the gap-filling step in uracil-initiated base excision repair in a bovine testis nuclear extract, J Biol Chem, 270, 949-957.

Srivastava, S., Z. Zou, K. Pirollo, W. Blattner and E. H. Chang. (1990) Gern-line transmission of a mutated p53 in a cancer-prone family with Li-Fraumeni syndrome, Nature (London), 348, 747-749.

Strauss, B. S. (1985) Translesion DNA synthesis: polymerase response to altered nucleotide, Cancer Surv., 4, 493-516.

Vandenbroucke, J. P., T. Koster, E. Briet, P. H. Reitsma, R. M. Bertina and F. R. Rosendaal. (1994) Increased risk of venous thrombosis in oral-contraceptive users who are carriers of factor V Leiden mutation, Lancet, 344, 1453-1457.

Vogelstein, B. and K. W. Kinzler. (1993) The multistep nature of cancer, Trends Genet., 9, 138-141.

Weeda, G., J. H. J. Hoeijmakers and D. Bootsma. (1993) Genes controlling nucleotide excision repair in eukaryotic cells, BioEssays, 15, 249-258.

Wei, Q., L. Cheng, W. K. Hong and M. R. Spitz. (1996) Reduced DNA repair capacity in lung cancer patients, Cancer Res., 56, 4103-4107.

Wei, Q., G. M. Matanoski, E. R. Farmer, M. A. Hedayati and L. Grossman. (1993) DNA repair and aging in basal cell carcinoma: a molecular epidemiology study, Proc. Natl. Acad. Sci. U S A, 90, 1614-1618.

Wei, Q., G. M. Matanoski, E. R. Farmer, M. A. Hedayati and L. Grossman. (1994) DNA repair and susceptibility to basal cell carcinoma: a case-control study, Am. J. Epidemiol., 140, 598-607.

Weinberg, R. A. (1989) Oncogenes, antioncogenes, and the molecular bases of multistep carcinogenesis, Cancer Res., 49, 3713-3721.

Wikman, H., A. Risch, F. Klimek, P. Schmezer, B. Spiegelhalder, H. Dienemann, K. Kayser, V. Schulz, P. Drings and H. Bartsch. (2000) hOGGl polymorphism and loss of heterozygosity (LOH): significance for lung cancer susceptibility in a caucasian population, Int. J. Cancer, 88, 932-937.

Wood, M. L., M. Dizdaroglu, E. Gajewski and J. M. Essigmann. (1990) Mechanistic studies of ionizing radiation and oxidative mutagenesis: Genetic effects of a single 8-hydroxyguanine (7-hydro-8-oxo-guanine) residue inserted at a unique site in a viral genome, Biochemistry, 29, 7024-7032.

What is claimed is:

1. A method of determining a risk of a subject to develop cancer, the method comprising determining a level of activity of a DNA repair /damage preventing enzyme in a tissue of the subject, and, according to said level, determining the risk of the subject to develop the cancer.

2. The method of claim 1, wherein said cancer is selected from the group consisting of lung cancer, blood cancers, colorectal cancer, breast cancer, prostate cancer, ovary cancer and head and neck cancer.

3. The method of claim 1, wherein said tissue is selected from the group consisting of blood cells, scraped cells and biopsies.

4. The method of claim 1, wherein said DNA repair/damage preventing enzyme is selected from the group consisting of a DNA N-glycosylase, deoxyribose phosphate lyase and AP endonuclease.

5. The method of claim 1, wherein said DNA N-glycosylase is selected from the group consisting of Uracil DNA glycosylase, hSMUG1, hMBD4, Mismatch-specific thymine/uracil glycosylase, Methylpurine DNA glycosylase, hNTH1, Adenine-specific mismatch DNA glycosylase and 8-oxoguanine DNA glycosylase.

6. The method of claim 1, wherein the risk is expressed as a fold risk increase as is compared to a normal, apparently healthy, population.

7. The method of claim 1, wherein the risk is expressed in enzymespecific activity units.

8. The method of claim 1, wherein the risk is expressed as a magnitude of a scale.

9. The method of claim 1, wherein determining the level of activity of the DNA repair enzyme is effected using a DNA substrate having at least one lesion therein.

10. The method of claim 9, wherein said at least one lesion is at a predetermined site in said DNA substrate.

11. The method of claim 9, wherein said substrate includes at least two different lesions of at least two types.

12. The method of claim 9, wherein said substrate includes a single lesion.

13. The method of claim 9, wherein said substrate includes at least two different lesions of a single type.

14. The method of claim 9, wherein said lesion is selected from the group consisting of uracil, 5-fluorouracil, 5-hydroxyuracil, isodialuric acid, alloxan, uracil or thymine in U/TpG:5meCpG, uracil (U:G), 3,N⁴-ethenocytosine, (eC:G), T (T:G), 3-methyladenine, 7-methyladenine, 3-methylguanine, 7-methylguanine, hypoxanthine, 1, N6-ethenoadenine, 1,N2-ethenoguanine, thymine glycol, cytosine glycol, dihydrouracil, formamidopyrimidine urea, adenine from A:G; A:8-oxoG; C:A, 2-hydroxyadenine, 2,5-amino-5-formamidopyrimidine, 7,8-dihydro-8-oxoguanine and abasic site.

15. The method of claim 1, wherein the subject is known to be, or is about to be, exposed to environmental conditions associated with increased risk of developing cancer.

16. A method of determining a risk of a subject to develop cancer, the method comprising determining:
(a) a presence or absence of exposure to environmental conditions associated with increased risk of developing cancer; and
(b) a level of activity of a DNA repair/damage preventing enzyme in a tissue of the subject; and according to said presence or absence and said level, determining the risk of the subject to develop the cancer.

17. The method of claim 16, wherein said cancer is selected from the group consisting of lung cancer, blood cancers, colorectal cancer, breast cancer, prostate cancer, ovary cancer and head and neck cancer.

18. The method of claim 16, wherein said tissue is selected from the group consisting of blood cells, scraped cells and biopsies.

19. The method of claim 16, wherein said DNA repair/damage preventing enzyme is selected from the group consisting of a DNA N-glycosylase, deoxyribose phosphate lyase and AP endonuclease.

20. The method of claim 16, wherein said DNA N-glycosylase is selected from the group consisting of Uracil DNA glycosylase, hSMUG1, hMBD4, Mismatch-specific thymine/uracil glycosylase, Methylpurine DNA glycosylase, hNTH1, Adenine-specific mismatch DNA glycosylase and 8-oxoguanine DNA glycosylase.

21. The method of claim 16, wherein the risk is expressed as a fold risk increase as is compared to a normal, apparently healthy, population.

22. The method of claim 16, wherein the risk is expressed in enzyme specific activity units.

23. The method of claim 16, wherein the risk is expressed as a magnitude of a scale.

24. The method of claim 16, wherein determining the level of activity of the DNA repair enzyme is effected using a DNA substrate having at least one lesion therein.

25. The method of claim 24, wherein said at least one lesion is at a predetermined site in said DNA substrate.

26. The method of claim 24, wherein said lesion is selected from the group consisting of uracil, 5-fluorouracil, 5-hydroxyuracil, isodialuric acid, alloxan, uracil or thymine in U/TpG:5meCpG, uracil (U:G), 3,N⁴-ethenocytosine, (eC:G), T (T:G), 3-methyladenine, 7-methyladenine, 3-methylguanine, 7-methylguanine, hypoxanthine, 1, N6-ethenoadenine, 1,N2-ethenoguanine, thymine glycol, cytosine glycol, dihydrouracil, formamidopyrimidine urea, adenine from A:G; A:8-oxoG; C:A, 2-hydroxyadenine, 2,5-amino-5-formamidopyrimidine, 7,8-dihydro-8-oxoguanine and abasic site.

27. The method of claim 24, wherein said substrate includes at least two different lesions of at least two types.

28. The method of claim 24, wherein said substrate includes a single lesion.

29. The method of claim 24, wherein said substrate includes at least two different lesions of a single type.

30. The method of claim 16, wherein said environmental conditions are selected from the group consisting of smoking and occupational exposure to smoke or ionizing radiation.

31. A method of predicting the efficacy of a mutagenic anti-cancer treatment in a subject, the method comprising determining a level of activity of a DNA repair/damage preventing enzyme in a tissue of the subject, and, according to said level, predicting the efficacy of the mutagenic anti-cancer treatment in the subject.

32. The method of claim 31, wherein said mutagenic anti-cancer treatment is selected from the group of chemotherapy and radiotherapy.

33. A method of selecting dosage of a mutagenic anti-cancer treatment for treating a subject, the method comprising determining a level of activity of a DNA repair/damage preventing enzyme in a tissue of the subject, and, according to said level, selecting dosage of the mutagenic anti-cancer treatment for treating the subject.

34. The method of claim 33, wherein said mutagenic anti-cancer treatment is selected from the group of chemotherapy and radiotherapy.

35. A kit for determining a level of activity of a DNA repair enzyme in a tissue of a subject, the kit comprising, a package including, contained in sealable containers, a DNA substrate having at least one lesion therein and a reaction buffer.

36. The kit of claim 35, further comprising test tubes for separating lymphocytes.

37. The kit of claim 36, wherein said test tubes are prepackaged with an anti-coagulant.

38. The kit of claim 35, further comprising a liquid having a specific gravity selected effective in separating lymphocytes from red blood cells via centrifugation.

39. The kit of claim 35, further comprising a solution having osmolarity selected effective in lysing red blood cells.

40. The kit of claim 35, further comprising a protein extraction buffer.

41. The kit of claim 35, further comprising reagents for conducting protein determinations.

42. The kit of claim 35, further comprising a purified DNA repair enzyme.

43. The kit of claim 35, wherein said substrate includes at least two different lesions of at least two types.

44. The kit of claim 35, wherein said substrate includes a single lesion.

45. The kit of claim 35, wherein said substrate includes at least two different lesions of a single type.

46. An isolated DNA molecule, in a single or double stranded form, comprising at least two different lesions of at least two types.

47. An isolated DNA molecule, in a single or double stranded form, comprising a plurality of lesions of a plurality of types.

48. A method of determining a presence of correlation or non-correlation between an activity of at least one DNA repair/damage preventing enzyme and at least one cancer, the method comprising determining a level of activity of at least one DNA repair/damage preventing enzyme in tissue derived from a plurality of cancer patients and a plurality of apparently normal individuals, and, according to said level determining said correlation or non-correlation between said activity of said at least one DNA repair enzyme and said at least one cancer.

49. The method of claim 48, wherein said cancer is selected from the group consisting of lung cancer, blood cancers, colorectal cancer, breast cancer, prostate cancer, ovary cancer and head and neck cancer.

50. The method of claim 48, wherein said tissue is selected from the group consisting of blood cells, scraped cells and biopsies.

51. The method of claim 48, wherein said DNA repair/damage preventing enzyme is selected from the group consisting of a DNA N-glycosylase, deoxyribose phosphate lyase and AP endonuclease.

52. The method of claim 48, wherein said DNA N-glycosylase is selected from the group consisting of Uracil DNA glycosylase, hSMUG1, hMBD4, Mismatch-specific thymine/uracil glycosylase, Methylpurine DNA glycosylase, HNTHl, Adenine-specific mismatch DNA glycosylase and 8-oxoguanine DNA glycosylase.

53. The method of claim 48, wherein the correlation or non-correlation is expressed in P values.

54. The method of claim 48, wherein determining the level of activity of the DNA repair enzyme is effected using a DNA substrate having at least one lesion therein.

55. The method of claim 54, wherein said at least one lesion is at a predetermined site in said DNA substrate.

56. The method of claim 54, wherein said substrate includes at least two different lesions of at least two types.

57. The method of claim 54, wherein said substrate includes a single lesion.

58. The method of claim 54, wherein said substrate includes at least two different lesions of a single type.

59. The method of claim 54, wherein said lesion is selected from the group consisting of uracil, 5-fluorouracil, 5-hydroxyuracil, isodialuric acid, alloxan, uracil or thymine in U/TpG:5meCpG, uracil (U:G), 3,N⁴-ethenocytosine, (eC:G), T (T:G), 3-methyladenine, 7-methyladenine, 3methylguanine, 7-methylguanine, hypoxanthine, 1, N6-ethenoadenine, 1,N2-ethenoguanine, thymine glycol, cytosine glycol, dihydrouracil, formamidopyrimidine urea, adenine from A:G; A:8-oxoG; C:A, 2-hydroxyadenine, 2,5-amino-5-formamidopyrimidine, 7,8-dihydro-8-oxoguanine and abasic site.


SEQUENCE LISTING (2 sequences)

SEQ ID NO: 1 - DNA, 32 bp, Artificial Sequence
  synthetic oligonucleotide
ccggtgcatgacactgtgacctatcctcagcg

SEQ ID NO: 2 - DNA, 32 bp, Artificial Sequence
  synthetic oligonucleotide
cgctgaggataggtcacagtgtcatgcaccgg